SEQUENCE LISTING (2 sequences)

SEQ ID NO: 1 - RNA, 21 nt, synthetic construct
  source 1..21: mol_type = other RNA
  modified_base order(1,3,5,7,9..11,13,15,17,19,21): mod_base = OTHER; note = 2-Prime-fluoro ribonucleosides
  modified_base order(2,4,6,8,12,14,16,18,20): mod_base = OTHER; note = 2-Prime-O-methyl ribonucleosides
  modified_base 1..3: mod_base = OTHER; note = Phosphorothioate linkage sodium salt
  modified_base 3..21: mod_base = OTHER; note = Phosphodiester linkage sodium salt
ggttaacaccatttacttcaa

SEQ ID NO: 2 - RNA, 23 nt, synthetic construct
  source 1..23: mol_type = other RNA
  modified_base order(2,4,6,8,10,14,16,18,20): mod_base = OTHER; note = 2-Prime-fluoro ribonucleosides
  modified_base order(1,3,5,7,9,11..13,15,17,19,21..23): mod_base = OTHER; note = 2-Prime-O-methyl ribonucleosides
  modified_base 1..3: mod_base = OTHER; note = Phosphorothioate linkage sodium salt
  modified_base 21..23: mod_base = OTHER; note = Phosphorothioate linkage sodium salt
  modified_base 3..21: mod_base = OTHER; note = Phosphodiester linkage sodium salt
ttgaagtaaatggtgttaaccag

(19) United States
(12) Patent Application Publication
(10) Pub. No.: US 2024/0000744 A1
(43) Pub. Date: Jan. 4, 2024

(54) Treatment of Hemophilia with Fitusiran

(71) Applicant: Genzyme Corporation, Cambridge, MA (US)

(72) Inventors: Shauna Andersson, Lexington, MA (US); Baisong Mei, Waban, MA (US); Fadi Shammas, Laval (CA); Christian Sussebach, Frankfurt am Main (DE)

(73) Assignee: Genzyme Corporation, Cambridge, MA (US)

(21) Appl. No.: 18/331,748

(22) Filed: Jun. 8, 2023

Related U.S. Application Data
(60) Provisional application No. 63/489,611, filed on Mar. 10, 2023.
(60) Provisional application No. 63/483,700, filed on Feb. 7, 2023.
(60) Provisional application No. 63/479,337, filed on Jan. 10, 2023.
(60) Provisional application No. 63/386,491, filed on Dec. 7, 2022.
(60) Provisional application No. 63/382,227, filed on Nov. 3, 2022.
(60) Provisional application No. 63/359,695, filed on Jul. 8, 2022.
(60) Provisional application No. 63/350,398, filed on Jun. 8, 2022.

(51) Int. Cl. A61K 31/351; A61P 7/04

(52) U.S. Cl. CPC A61K 31/351; A61P 7/04

(57) ABSTRACT

The present disclosure provides methods for using fitusiran to treat patients with hemophilia A or hemophilia B.

### CROSS-REFERENCE TO RELATED APPLICATIONS

The present application claims priority from U.S. Applications 63/350,398, filed Jun. 8, 2022; 63/359,695, filed Jul. 8, 2022; 63/382,227, filed Nov. 3, 2022; 63/386,491, filed Dec. 7, 2022; 63/479,337, filed Jan. 10, 2023; 63/483,700, filed Feb. 7, 2023; and 63/489,611, filed Mar. 10, 2023. The contents of the priority applications are incorporated by reference herein in their entirety.

### SEQUENCE LISTING

The instant application contains a Sequence Listing which has been submitted electronically in XML format and is hereby incorporated by reference in its entirety. Said XML copy, created on Jun. 2, 2023, is named 122548WO020.xml and is 8,023 bytes in size.

### BACKGROUND OF THE INVENTION

Hemophilia A and hemophilia B are X-linked recessive inherited bleeding disorders, characterized by deficiency of coagulation Factor VIII (FVIII) or Factor IX (FIX), leading to a profound defect of thrombin generation with impaired hemostasis and increased risk of bleeding. Hemophilia A is found in approximately 1 in 4,000 males whereas hemophilia B is five times less common and seen in approximately 1 in 20,000 males. The disease phenotype presents similarly in hemophilia A and B.

Hemophilia is classified as mild (factor levels 6% to 30%), moderate (factor levels 1% to 5%), or severe (factor levels <1%) based on clotting factor activity relative to normal (healthy, non-hemophiliac plasma levels of factor are 50% to 150%). Patients with mild hemophilia typically experience bleeding after a serious injury or surgery. Patients with moderate hemophilia experience bleeding episodes associated with injuries, and may have spontaneous bleeding episodes. Severe hemophilia patients experience substantial bleeding with injury and may have frequent spontaneous bleeding episodes resulting in debilitating musculoskeletal damage that can markedly impair a patient's mobility and quality of life (QoL).

The hemostatic system aims to maintain the integrity of the vasculature by protecting against bleeding from vessel lesions combined with multiple options to prevent thrombosis. This hemostatic balance is achieved through an orchestrated regulation of both procoagulant (e.g., Factor V (FV), Factor VII (FVII), FVIII, FIX, Factor X (FX)) and anticoagulant (e.g., antithrombin, protein C/protein S and tissue factor pathway inhibitor) factors. Recent studies have suggested that coinheritance of a deficiency in natural anticoagulants may contribute to a milder phenotype in patients with hemophilia.

Antithrombin (AT) is a liver-expressed natural anticoagulant that plays a key role in inhibiting thrombin. AT acts as an inhibitor of Factor VIIa and Factor Xa, which are typically at normal levels in patients with hemophilia A or B. Extensive preclinical in vitro and in vivo studies have described reduction of AT as a potential safe and effective way to correct thrombin generation in both hemophilia A and B and control against microvascular and macrovascular traumatic bleeding episodes. Therefore, suppression of AT production is being investigated as a potential hemophilia treatment.

Replacement with factor concentrates is the current standard of care for hemophilia patients without inhibitory antibodies to FVIII or FIX. While the current standard of care of factor substitution therapy, administered either episodically or as routine prophylaxis, is well established, safe, and efficacious, it is associated with high treatment burden due to requiring intravenous (IV) administration on a frequent schedule (2 to 3 times per week or more) to prophylactically maintain hemostasis. Factor concentrates also can be of limited availability in developing nations. In addition, patients receiving factor concentrates may develop an inhibitory antibody to factor, a result that carries a poorer prognosis and requires a change in treatment regimen to infusion with bypassing agents (BPAs).

Hemophilia patients who develop inhibitory antibodies to factor concentrates represent a distinct subset of the population; these patients typically experience more difficult-to-treat bleeding episodes, leading to increased morbidity and increased mortality. Development of inhibitors to infused factor occurs mainly in severe hemophilia, and more frequently in hemophilia A (up to 39% of patients) than in hemophilia B (1% to 3.5% of patients) with the greatest risk of development in the early exposure days. These “inhibitor” patients, may be eligible for immune tolerance therapy; however, in most cases bleeding episodes require hemostatic intervention with intravenously (IV)-administered BPAs, i.e., recombinant Factor VIIa (rVIIa), or activated prothrombin complex concentrates (aPCC), either as prophylaxis or as on-demand episodic treatment of bleeding episodes.

Thus, there remains a need for alternative treatments for subjects having hemophilia, such as a subcutaneous therapy that can effectively and safely prevent or reduce the frequency of bleeding episodes in patients with hemophilia A or B, including those with inhibitors, while reducing treatment burden, improving clinical outcomes, and enhancing quality of life.

### SUMMARY OF THE INVENTION

The present disclosure provides methods of prophylactically treating hemophilia A or B with or without inhibitors with fitusiran. The patients herein have previously been on a different prophylaxis. In some embodiments, the present methods reduce the annual bleeding rate (ABR), the annual spontaneous bleed rate (AsBR), and/or the annual joint bleed rate (AjBR), and/or improves patient-reported outcomes (e.g., quality of life) in a human subject having hemophilia A or B with or without inhibitors who has been on prophylactic treatment with a replacement factor or a BPA. In some embodiments, the methods comprise subcutaneously administering to the human subject in need thereof a therapeutically effective amount of fitusiran and terminating the prophylactic replacement factor or BPA treatment in the subject within about two months, about one month, or optionally within about 28 or about seven days, of the first dose of fitusiran. In some embodiments, the methods comprise subcutaneously administering to the human subject in need thereof a therapeutically effective amount of fitusiran. In some embodiments, the methods reduce the annualized weight-adjusted consumption of replacement factor or BPA in the patient, the total weight-adjusted dose of replacement factor/BPA in the patient, the mean consumption of replacement factor/BPA in a patient over a given period of time, the number of injections of replacement factor/BPA required to treat a breakthrough bleed in a patient, or the number of breakthrough bleeds requiring treatment over a given period of time.

Also provided herein is fitusiran for use in these treatment methods, use of fitusiran in the manufacture of a medicament for treating hemophilia A or B with or without inhibitors in a method herein, and a pharmaceutical composition comprising fitusiran for use in the present treatment methods.

Other features, objectives, and advantages of the invention are apparent in the detailed description that follows. It should be understood, however, that the detailed description, while indicating embodiments and aspects of the invention, is given by way of illustration only, not limitation. Various changes and modification within the scope of the invention will become apparent to those skilled in the art from the detailed description.

### BRIEF DESCRIPTION OF THE FIGURES

FIG. 1 shows the expanded structural formula, chemical formula, and molecular mass of fitusiran (sodium form).

FIG. 2 shows the clinical trial study design for patients who are treated prophylactically with replacement factor or BPA before beginning fitusiran treatment. AT: antithrombin. For subgroup of Cohort A patients enrolling directly into the fitusiran treatment period, see FIG. 3. “a”: patients will continue to receive prescribed factor or BPA prophylaxis for the first 7 days of the onset period. “b”: following final fitusiran dose, AT activity level will be monitored at monthly intervals following the final fitusiran dose until activity levels return to approximately 60% (per the central laboratory) or per Investigator discretion in consultation with the study Medical Monitor.

FIG. 3 shows the clinical trial study design for hemophilia B patients with inhibitory antibodies to Factor IX who are also not responding adequately to BPA prophylaxis treatment (historical ABR≥20), and are therefore not currently undergoing prophylactic treatment with BPA. These patients directly begin fitusiran treatment without participating in the six-month factor/BPA period described in FIG. 2. “a”: patients will receive prescribed factor or BPA prophylaxis for the first 7 days of the onset period. “b”: same meaning as in FIG. 2.

FIG. 4 shows the median ABR in patients treated prophylactically with fitusiran or factor/BPA.

FIG. 5 shows the AjBR and AsBR in patients treated prophylactically with fitusiran or factor/BPA.

FIG. 6 shows the health-related quality of life burden for all clinical trial patients six months prior to the start of the study (i.e., at month −6).

FIG. 7 shows the Haem-A-QoL scores in patients treated prophylactically with fitusiran or factor/BPA.

FIG. 8 shows transformed Haem-A-QoL scores in patients treated prophylactically with fitusiran or factor/BPA.

FIG. 9 shows the TSQM-9 scores in patients treated prophylactically with fitusiran or factor/BPA.

FIG. 10 shows TSQM-9 scores in patients treated prophylactically with fitusiran or factor/BPA.

FIG. 11 is a chart showing the mean AT levels in patients treated with fitusiran.

FIG. 12 is a chart showing the mean change in peak thrombin generation (TG) in patients treated prophylactically with fitusiran.

FIG. 13 is a bar graph showing bleeding events (median observed ABR, AjBR, and AsBR) in adolescents enrolled in the clinical trial.

FIG. 14 is a bar graph showing the mean change in Haemo-QoL transformed total score from baseline in adolescents enrolled in the clinical trial. For fitusiran 80 mg prophylaxis, n=14.

FIG. 15 is pre-study survey regarding the impact of hemophilia and its treatments on patients. ^{a}Percentage based on the 19 participants asked this question; ^{b}Percentage based on the 22 participants asked this question; ^{c}Percentage based on the 20 participants asked this question; ^{d}Percentage based on the 18 participants asked this question; ^{e}Percentage based on the 16 participants asked this question.

FIG. 16 is a bar graph showing the degree and importance of improvements in patients treated prophylactically with fitusiran.

FIG. 17 shows the participant satisfaction rating in patients treated prophylactically with fitusiran.

### DETAILED DESCRIPTION OF THE INVENTION

The present disclosure features methods of using fitusiran for routine prophylaxis to reduce the frequency of bleeding episodes in adult and adolescent patients (212 years old) with hemophilia, such as hemophilia A (congenital factor VIII deficiency) or hemophilia B (congenital factor IX deficiency), with or without inhibitors. These methods reduce the frequency of total bleeds, including spontaneous and joint bleeds, compared to the frequency of bleeds in patients who are treated prophylactically with replacement factor or BPAs instead of fitusiran. These methods also result in an improvement in patient-reported outcomes (PROs) and quality of life (QoL) compared to the PROs and QoL in patients treated prophylactically with replacement factor or BPAs.

In some embodiments, the patient is an adolescent patient (i.e., a patient that is 12-17 years of age, inclusive). Adolescence is associated with significant physical, psychological, and social changes. Hemophilia can present additional challenges for adolescents as they may be prevented from experiencing the same social and physically active life as their peers (see, e.g., Hoefnagels et al., Patient Prefer Adherence (2020) 14: 163-71).

A hemophilia A or B patient with inhibitors refers to a patient who has developed alloantibodies to the factor he/she has previously received (e.g., factor VIII for hemophilia A patients or factor IX for hemophilia B patients). A hemophilia A or B patient with inhibitors may become refractory to replacement coagulation factor therapies. A patient without inhibitors refers to a patient who does not have such alloantibodies. The present treatment methods may be beneficial for hemophilia A patients with inhibitors, as well as for hemophilia B patients with inhibitors. As used herein, “hemophilia A or B with inhibitors” refers to hemophilia A with inhibitors, or hemophilia B with inhibitors. As used herein, a patient refers to a human patient.

This invention is based in part on the finding that patients treated prophylactically with fitusiran have improved outcomes (e.g., reduced bleeding rates and improved quality of life) as compared to patients given standard-of-care prophylactic treatment (replacement factor or BPA). The clinical trial protocol and results disclosed herein are the first direct comparison of these two treatment regimens in hemophilia A and B patients with or without inhibitors.

In some embodiments, the mechanism of action and formulation of fitusiran allows for only 6-12 subcutaneous injections per year to achieve consistent bleed protection. As a subcutaneously administered drug, fitusiran has the benefits of reduced administration time, less pain due to reduced needle size and volume, and less equipment and training required compared to intravenously administered drugs. Fewer bleeds and reduced overall treatment and disease burden with fitusiran prophylaxis may improve quality of life for people with hemophilia.

### I. Fitusiran Pharmaceutical Compositions

Hemophilia results in a profound defect in thrombin generation, and further, hemophilia severity is correlated with the inability to generate thrombin. Without being bound by theory, it is believed that fitusiran-mediated lowering of antithrombin (AT) levels will increase thrombin generation and thus improve hemostasis in patients with hemophilia. Antithrombin is encoded by the SERPINCI gene.

Fitusiran, whose structure is described herein, is a synthetic, chemically modified double-stranded small interfering RNA (siRNA) oligonucleotide covalently linked to a tri-antennary N-acetyl-galactosamine (GalNAc) ligand targeting the AT3 mRNA in the liver, thereby suppressing the synthesis of antithrombin. The nucleosides in each strand of fitusiran are connected through either 3′-5′ phosphodiester or phosphorothioate linkages, thus forming the sugar-phosphate backbone of the oligonucleotide.

The sense strand and the antisense strand of fitusiran contain 21 and 23 nucleotides, respectively. The 3′-end of the sense strand is conjugated to the GalNAc containing moiety (referred to as L96) through a phosphodiester linkage. The sense strand contains two consecutive phosphorothioate linkages at its 5′ end. The antisense strand contains four phosphorothioate linkages, two at the 3′ end and two at the 5′ end. The 21 nucleotides of the sense strand hybridize with the complementary 21 nucleotides of the antisense strand, thus forming 21 nucleotide base pairs and a two-base overhang at the 3′-end of the antisense strand. See also U.S. Pat. Nos. 9,127,274, 11,091,759, and WO 2019/014187.

The two nucleotide strands of fitusiran are shown below:
sense strand: 5′Gf-ps-Gm-ps-Uf-Um-Af-Am-Cf-Am-Cf-Cf-Af-Um-Uf-Um-Af-Cm-Uf-Um-Cf-Am-Af-L96 3′ (SEQ ID NO:1), and
antisense strand: 5′ Um-ps-Uf-ps-Gm-Af-Am-Gf-Um-Af-Am-Af-Um-Gm-Gm-Uf-Gm-Uf-Um-Af-Am-Cf-Cm-ps-Am-ps-Gm 3′ (SEQ ID NO:2),
   wherein
Af=2′-fluoroadenosine (i.e., 2′-deoxy-2′-fluoroadenosine)
Cf=2′-fluorocytidine (i.e., 2′-deoxy-2′-fluorocytidine)
Gf=2′-fluoroguanosine (i.e., 2′-deoxy-2′-fluoroguanosine)
Uf=2′-fluorouridine (i.e., 2′-deoxy-2′-fluorouridine)
Am=2′-O-methyladenosine
Cm=2′-O-methylcytidine
Gm=2′-O-methylguanosine
Um=2′-O-methyluridine
“-” (hyphen)=3′-5′ phosphodiester linkage sodium salt
“-ps-”=3′-5′ phosphorothioate linkage sodium salt and wherein L96 has the following formula:

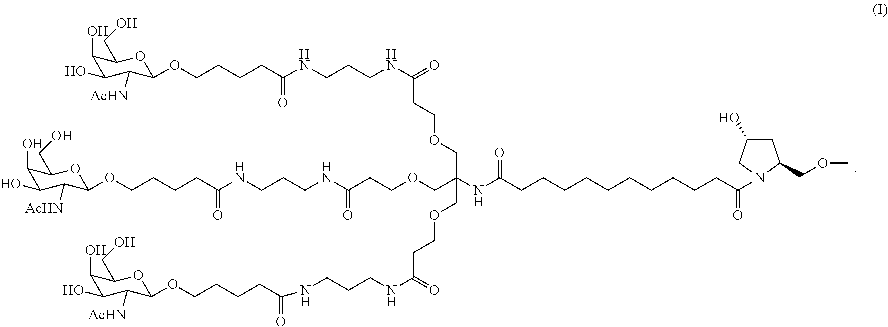 [SMILES: COC[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

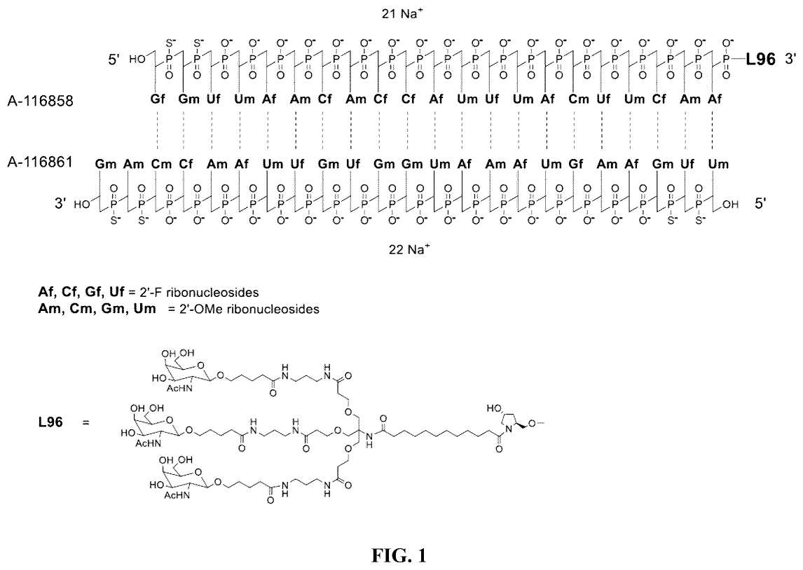

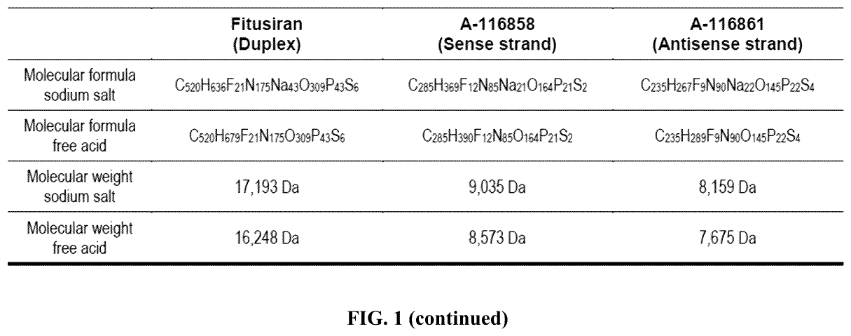

The expanded structural formula, molecular formula, and molecular weight of fitusiran (sodium form) are shown in FIG. 1. As used herein, the term 2′-fluoroadenosine is used interchangeably with the term 2′-deoxy-2′-fluoroadenosine; the term 2′-fluorocytidine is used interchangeably with the term 2′-deoxy-2′-fluorocytidine; the term 2′-fluoroguanosine is used interchangeably with the term 2′-deoxy-2′-fluoroguanosine, and the term 2′-fluorouridine is used interchangeably with the term 2′-deoxy-2′-fluorouridine.

The structure of fitusiran can also be described using the following diagram, wherein the X is O:

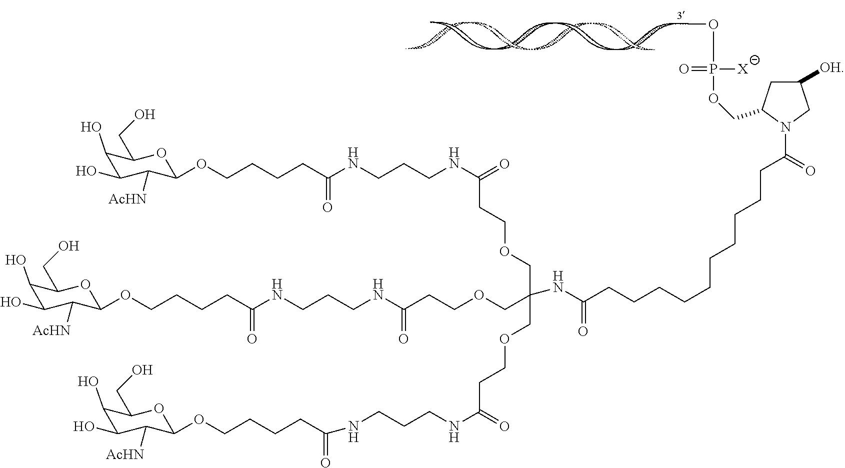

For use in the present treatment methods, fitusiran may be provided in a pharmaceutical composition comprising it and a pharmaceutically acceptable excipient. In certain embodiments, fitusiran is in sodium salt form.

In some embodiments, fitusiran is provided in an aqueous solution at a concentration of about 1 to about 200 mg/mL (e.g., about 50 to about 150 mg/mL, about 80 to about 110 mg/mL, or about 90 to about 110 mg/mL). As used herein, values intermediate to recited ranges and values are also intended to be part of this disclosure. In addition, ranges of values using a combination of any of recited values as upper and/or lower limits are intended to be included. In further embodiments, the pharmaceutical composition comprises fitusiran at a concentration of about 6.25, about 12.5, about 25, about 50, about 75, about 100, about 125, about 150, or about 200 mg/mL.

Unless otherwise indicated, a fitusiran weight recited in the present disclosure is the weight of fitusiran free acid (the active moiety), even though fitusiran is injected to patients subcutaneously in its sodium form (in an aqueous solution). For example, 100 mg/mL fitusiran means 100 mg of fitusiran free acid (equivalent to 106 mg fitusiran sodium, the drug substance) per mL.

In some embodiments, the pharmaceutical compositions comprise fitusiran in a phosphate-buffered saline. The phosphate concentration in the solution may be about 1 to 10 mM (e.g., 2, 3, 4, 5, 6, 7, 8, or 9 mM), with a pH of 6.0-8.0. The pharmaceutical compositions herein may include a preservative such as EDTA. Alternatively, the pharmaceutical compositions are preservative-free. In particular embodiments, the fitusiran pharmaceutical composition is preservative-free and comprises, consists of, or consists essentially of about 100 mg of fitusiran per mL of a 5 mM phosphate buffered saline (PBS) solution. The PBS solution is composed of sodium chloride, dibasic sodium phosphate (heptahydrate), and monobasic sodium phosphate (monohydrate). Sodium hydroxide solution and diluted phosphoric acid may be used to adjust the pH of the composition to 7.0.

The pharmaceutical composition may be provided in a container (e.g., a vial or a syringe). The container may contain single or multiple doses. In some embodiments, the container is a single-use container (e.g., a single-use ampule or a single-use syringe such as a single-use pre-filled syringe), with each container containing about 10 to about 100 mg fitusiran (e.g., about 10 mg, about 20 mg, about 25 mg, about 40 mg, about 50 mg, or about 80 mg). The fitusiran may be provided in a solid form in the container and reconstituted in an aqueous solution (e.g., PBS) prior to use, with the reconstituted solution containing about 1 to about 150 mg/mL (e.g., 100 mg/mL) fitusiran. In some embodiments, fitusiran is provided in sodium salt form in a single-use glass vial or a single-use prefilled syringe (e.g., one with a safety system). In further embodiments, each vial or syringe contains about 80 mg of fitusiran in about 0.8 mL (or about 50 mg of fitusiran in about 0.5 mL, about 20 mg of fitusiran in about 0.2 mL, or about 10 mg of fitusiran in about 0.1 mL) of 5 mM phosphate buffered saline solution (pH 7.0); and the solution is administered to patients through subcutaneous injection. The solution can be stored at 2 to 30° C. (e.g., 2 to 8° C.).

In particular embodiments, the fitusiran composition for subcutaneous injection contains fitusiran in a 5 mM phosphate buffered saline having 0.64 mM NaH₂PO₄, 4.36 mM Na₂HPO₄, and 84 mM NaCl at pH 7.0. In certain embodiments, the composition of fitusiran solution for subcutaneous injection is shown in Table 1 below:

TABLE 1
Exemplary Formulation

| | Composition | | | |
|---|---|---|---|---|
| | | | Per unit | Per unit |
| | | | (2 mL | (1 mL |
| | Percentage | Per ml | vial) | syringe) |
| Components | [%] | [mg] | [mg] | [mg] |
| Fitusiran (active moiety) | 10 | 100 [106] | 80 [84.8] | 80 [84.8] |
| [equivalent to fitusiran sodium] | | | | |
| Sodium chloride | 0.49 | 4.909 | 3.927 | 3.927 |
| Dibasic sodium phosphate | 0.12 | 1.169 | 0.935 | 0.935 |
| (heptahydrate) | | | | |
| Monobasic sodium phosphate | <0.01 | 0.0885 | 0.0708 | 0.0708 |
| (monohydrate) | | | | |
| Phosphoric acid, concentrated | — | q.s. pH 7.0 | q.s. pH 7.0 | q.s. pH 7.0 |
| Sodium hydroxide | — | q.s. pH 7.0 | q.s. pH 7.0 | q.s. pH 7.0 |
| Water for injection | q.s. 100 | q.s. 1 mL | q.s. 0.8 mL | q.s. 0.8 mL |

q.s.: quantum satis.

While the fitusiran dosage weight described herein refers to the weight of fitusiran free acid (active moiety), administration of fitusiran to patients herein refers to administration of fitusiran sodium (drug substance) provided in a pharmaceutically suitable aqueous solution (e.g., a phosphate-buffered saline at a physiological pH).

### II. Therapeutic Use of Fitusiran

Fitusiran can suppress liver production of antithrombin (AT). In its role as an anti-coagulant, AT regulates hemostasis by directly targeting thrombin production or by inactivating uncomplexed FXa, which in turn reduces thrombin production (Quinsey et al., Int J Biochem Cell Biol. (2004) 36(3):386-9). Fitusiran may be used to treat those who have impaired hemostasis. For example, fitusiran can be used to treat patients with hemophilia A or B with or without inhibitors for routine prophylaxis to prevent or reduce the frequency of bleeding episodes. In particular embodiments, fitusiran is used to treat adult and adolescent patients (212 years of age) with hemophilia A or B (congenital factor VIII or factor IX deficiency) with or without inhibitors. The present methods include administering to the hemophilia patient (e.g., a hemophilia A or B patient) in need thereof a therapeutically effective amount of fitusiran. “Therapeutically effective amount” refers to the amount of fitusiran that helps the patient to achieve a desired clinical endpoint.

In some embodiments, a patient with hemophilia A or B with or without inhibitors is treated with a subcutaneous dose of fitusiran at about 50 mg per dose about every two months (or about every eight weeks). In other embodiments, a patient with hemophilia A or B with or without inhibitors is treated with a subcutaneous dose of fitusiran at about 50 mg about every month (or about every four weeks). In yet other embodiments, a patient with hemophilia A or B with or without inhibitors is treated with a subcutaneous dose of fitusiran at about 80 mg about every two months (or about every eight weeks). In yet other embodiments, a patient with hemophilia A or B with or without inhibitors is treated with a subcutaneous dose of fitusiran at about 80 mg about every month (or about every four weeks). In yet other embodiments, a patient with hemophilia A or B with or without inhibitors is treated with a subcutaneous dose of fitusiran at about 20 mg about every two months (or about every eight weeks). In yet other embodiments, a patient with hemophilia A or B with or without inhibitors is treated with a subcutaneous dose of fitusiran at about 20 mg about every month (or about every four weeks). In yet other embodiments, a patient with hemophilia A or B with or without inhibitors is treated with a subcutaneous dose of fitusiran at about 10 mg about every month (or about every four weeks).

### III. Administration of Fitusiran Pharmaceutical Compositions

The fitusiran pharmaceutical composition may be administered by any means known in the art including, but not limited to, intraperitoneal, intravenous, intramuscular, subcutaneous, transdermal, or hepatic portal vein administration. In certain embodiments, the pharmaceutical composition is administered by subcutaneous injection at a dose strength of, for example, about 10 to about 100 mg (e.g., about 10 to about 95 mg, about 40 to about 90 mg, about 50 to about 100 mg, about 50 to about 90 mg, about 50 to about 85 mg, or about 50 to about 80 mg) per dose. In particular embodiments, fitusiran is administered subcutaneously at about 10, about 20, about 50, or about 80 mg (weight of active moiety) per dose in a PBS solution as described above.

A plurality of fitusiran doses may be administered to a subject at an interval of about 1, about 2, about 3, about 4, about 5, about 6, about 7, or about 8 weeks, or of about 1, about 2, or about 3 months. In particular embodiments, a fixed dose of fitusiran (e.g., about 50 or about 80 mg subcutaneous injection) is administered to a hemophilia patient (e.g., a hemophilia A or hemophilia B patient who is twelve years or older and who has or has not developed inhibitors) about once every four weeks or about once a month or about once every eight weeks or about once every other month.

In some embodiments, the present pharmaceutical compositions can be administered with other pharmaceuticals and/or other therapeutic methods, such as those that are currently employed for treating bleeding disorders. For example, in certain embodiments, fitusiran is administered in combination with a second agent useful in treating hemophilia A and/or B. Examples of such second agents are fresh-frozen plasma (FFP); rFVIIa; aPCC; recombinant or plasma-derived FVIII or FIX; virus-inactivated, vWF-containing FVIII concentrates; desensitization therapy which may include large doses of FVIII or FIX, along with steroids or intravenous immunoglobulin (IVIG) and cyclophosphamide; plasmapheresis in conjunction with immunosuppression and infusion of FVIII or FIX, with or without antifibrinolytic therapy; immune tolerance induction (ITI), with or without immunosuppressive therapy (e.g., cyclophosphamide, prednisone, and/or anti-CD20); desmopressin acetate (DDAVP); antifibrinolytics, such as aminocaproic acid and tranexamic acid; antihemophilic agents; corticosteroids; immunosuppressive agents; and estrogens. The fitusiran composition and the additional therapeutic agent and/or treatment may be administered at the same time and/or in the same combination, e.g., parenterally, or the additional therapeutic agent can be administered as part of a separate composition or at separate times and/or by another method known in the art or described herein.

IV. Treatment with Replacement Factor or BPA

The standard of care for hemophilia A and B treatment is routine prophylactic use of replacement factor or bypassing agent (BPA). In some embodiments, patients treated with the current treatment methods have previously undergone prophylactic treatment with factor or bypassing agent. Patients undergoing prophylactic treatment with replacement factor or BPA are treated as described in Table 2. Factor and bypassing agents are administered intravenously.

TABLE 2
Factor or bypassing agent prophylaxis requirements

| Type of Factor or | Frequency of |
|---|---|
| BPA Replacement | Administration |
| Standard half-life FVIII | Twice weekly |
| Extended half-life FVIII | Once weekly |
| Standard half-life FIX | Once weekly |
| Extended half-life FIX | Once biweekly |
| aPCC | Twice weekly |
| rFVIIa | Every other day |

In some embodiments, hemophilia patients with inhibitors (e.g., hemophilia B patients with inhibitors) do not respond adequately to BPA prophylaxis treatment are therefore not treated prophylactically with BPA.

In some embodiments of the present invention, patients prophylactically treated with fitusiran have been previously prophylactically treated with replacement factor or BPA. The first 28 days of fitusiran treatment is referred to as the onset period. During the onset period, AT lowering will be progressing toward therapeutic levels. During the onset period, patients continue factor or BPA prophylaxis with minimum frequency as in Table 2 for the first seven days. Subsequent to Day 7 of the fitusiran treatment period, factor concentrates or BPAs are administered only for bleeding episodes or if needed in advance of invasive medical procedures.

Bleeding Episode Management Recommendations

During the Factor or Bypassing Agent Prophylaxis Period: Bleeding management therapy with factors or BPAs are managed based on the local standard practice for treating hemophilia patients.

During the Fitusiran Treatment Period: Given the mechanism of action and pharmacodynamics profile of fitusiran, the factor or BPA dose necessary to safely and effectively treat breakthrough bleeding episodes in patients receiving fitusiran during the fitusiran treatment period will be lower than standardly prescribed. After administration of fitusiran, AT lowering will be progressing toward therapeutic levels. As quickly as seven days after the initial fitusiran dose, the majority of patients will have AT levels at or below 60% residual activity. By 14 days after dosing, it is expected that 94% of patients will have AT lowering of >50%, with a median value of 66.8%. Based on these AT kinetics, it is recommended that patients continue with their standard factor or BPA regimens for the first days following initiation of fitusiran dosing, with institution of the protocol-specific bleed management guidelines with reduced factor or BPA at Day 8 and beyond (Table 3).

TABLE 3
Bleed Management Dosing Guidelines by Specific Product

| | | Factor IX | Factor IX | | Recombinant |
|---|---|---|---|---|---|
| | Factor | Standard | Extended | | Factor |
| | VIII | Half-Life | Half-Life | aPCC | VIIa |
| Recommended | 10 IU/kg | 20 IU/kg | 20 IU/kg | 30 U/kg | ≤45 μg/kg |
| Single dose of | | | | | |
| Single dose | 20 IU/kg | 30 IU/kg | 30 IU/kg | 50 U/kg | 45 μg/kg |
| should not | | | | | |
| exceed | | | | | |
| | Should not | Should not | Should not | Should not | Should not |
| | repeat in | repeat in | repeat in | repeat in | repeat in |
| | less than | less than | less than | less than | less than |
| | 24 hours | 24 hours | 5-7 days | 24 hours | 2 hours |

Importantly, after Day 7 of the fitusiran treatment period, patients do not use factor, BPA, or other hemostatic agents as prophylaxis for bleeding episode prevention, including doses related to anticipated hemostatic challenges such as physical activity.

Day 1 to Day 7 of Fitusiran Treatment Period: Patients continue with their standard factor or BPA regimens.

Day 8 and Beyond of Fitusiran Treatment Period: When a patient experiences a symptom that may be consistent with bleeding episodes, the following steps are followed:
A single dose is administered according to the guidelines in Table 3.
The patient is instructed to re-evaluate symptoms in 24 hours for bleeds treated with FVIII, FIX or aPCC and in 2-3 hours for bleeds treated with rFVIIa.
   a. Administration of FIX Extended half-life is not be more frequent than every 5-7 days.
Doses are not administered at less than 24-hour intervals (except rFVIIa as indicated in Table 3).
Doses do not exceed the protocol maximum recommended dose indicated in Table 3.
Antifibrinolytics are not used in combination with factor or BPA.

Consumption of Factor/BPA in Patients Prophylactically Treated with Fitusiran

In some embodiments, the present treatment methods reduce the consumption of replacement factor/BPA in a patient, including the annualized weight-adjusted consumption of replacement factor or BPA in the patient. In some embodiments, the present treatment methods reduce the annualized weight-adjusted consumption of aPCC for bleed therapy by about 98.9%; the annualized weight-adjusted consumption of rFVIIa for bleed therapy by about 96.8%; the annualized weight-adjusted consumption of FVIII for bleed therapy by about 79.4%; and/or the annualized weight-adjusted consumption of FIX for bleed therapy by 93.8%.

In some embodiments, the present treatment methods reduce the number of treated breakthrough bleeds in a patient. In some embodiments, the present treatment methods reduce the total weight-adjusted dose of replacement factor/BPA, the mean consumption of replacement factor/BPA, or the number of injections of replacement factor/BPA required to treat a breakthrough bleed in a patient. The present treatment methods may decrease the number of patients that require replacement factor/BPA for breakthrough bleed treatment.

### V. Selection of Patients

The present treatments may be used to prophylactically treat patients with hemophilia A or hemophilia B. In some embodiments, the patient is a male aged 12 years with severe hemophilia A or B. A diagnosis of severe hemophilia is based on Factor VIII level of <1% (for hemophilia A) or a Factor IX level of C2% (for hemophilia B). In some embodiments, the patient has used replacement factors or bypassing agents prophylactically to manage bleeding episodes for at least six months prior to the start of treatment.

In some embodiments, the patient has inhibitory antibodies to Factor VIII or Factor IX (i.e., is a patient with inhibitors). A patient is defined as a patient with inhibitors if they meet at least one of the following Nijmegen-modified Bethesda assay results criteria:
a. inhibitor tier of ≥0.6 BU/mL prior to treatment, or
b. inhibitor titer of <0.6 BU/mL prior to treatment with medical record evidence of two consecutive titers ≥0.6 BU/mL, or
c. inhibitor titer of <0.6 BU/mL prior to treatment with medical record evidence of anamnestic response.

In some embodiments, the patient does not have inhibitory antibodies to Factor VIII or Factor IX (i.e., is a patient without inhibitors). A patient is defined as a patient without inhibitors if they meet at least one of the following Nijmegen-modified Bethesda assay results criteria:
a. Nijmegen-modified Bethesda assay inhibitor titer of <0.6 BU/mL prior to treatment,
b. no use of bypassing agents to treat bleeding episodes for at least the last six months prior to treatment, and
c. no history of immune tolerance induction therapy within the past three years prior to treatment.

In some embodiments, the patient has inhibitory antibodies to Factor VIII or Factor IX (Cohort A), and has experienced a minimum of two bleeding episodes requiring BPA treatment within the last six months prior to treatment. In some embodiments, the patient has inhibitory antibodies to Factor VIII or Factor IX, but is not treated prophylactically with a bypassing agent. In some embodiments, this patient has hemophilia B with inhibitory antibody to Factor IX as defined above, and does not respond adequately to BPA treatment (historical ABR ≥20) prior to fitusiran treatment.

In some embodiments, the patient does not have inhibitory antibodies to Factor VIII or Factor IX (Cohort B), and has experienced a minimum of one bleeding episode requiring factor treatment within the last 12 months prior to treatment.

In some embodiments, the patient has been prescribed (and adhered to) prophylactic treatment of hemophilia with factor concentrates or BPAs for at least six months prior to treatment. The prophylactic treatment regimen must be consistent with the approved prescribing information for the product or local recommendations, allowing for adjustment to individual patient response, and designed to decrease spontaneous bleeding.

In some embodiments, the patient does not have a known co-existing bleeding disorder other than hemophilia A or B, i.e., Von Willebrand's disease, additional factor deficiencies, or platelet disorders. In some embodiments, the patient is not currently participating in immune tolerance induction therapy (ITI). The patient may not have an AT activity <60% prior to treatment. In some embodiments, the patient does not have a clinically significant liver disease, as indicated by (i) INR>1.2; ALT and/or AST>1.5×upper limit of normal reference range (ULN); (ii) total bilirubin >ULN (>1.5×ULN in patients with Gilbert's Syndrome); (iii) history of portal hypertension, esophageal varices, or hepatic encephalopathy; or (iv) presence of ascites by physical exam.

In some embodiments, the patient is not Hepatitis C virus antibody positive. A patient who is Hepatitis C virus antibody positive may be treated with the present methods only if they have:
a. completed curative treatment at least 12 weeks prior to enrollment and attained sustained virologic response as documented by a negative HCV RNA prior to treatment, or they have spontaneously cleared infection as documented by negative HCV RNA prior to treatment, and
b. no evidence of cirrhosis according to FibroScan <12.5 kPa (where available), or FibroTest score <0.75 and APRI<2 (if FibroScan unavailable).

In some embodiments, the patient does not have acute hepatitis, i.e., hepatitis A or hepatitis E. In some embodiments, the patient does not have acute or chronic hepatitis B infection (IgM anti-HBc antibody positive or HBsAg positive). In some embodiments, the patient does not have (i) a platelet count ≤100,000/μL, (ii) is not HIV positive with CD4 count <200 cells/L, and/or (iii) does not have an estimated glomerular filtration rate ≤45 mL/min/1.73 m2 (using the Modification of Diet in Renal Disease [MDRD] formula).

The patient may not have a co-existing thrombophilic disorder, as determined by presence of any of the below:
a. FV Leiden mutation (homozygous or heterozygous),
b. protein S deficiency,
c. protein C deficiency, or
d. prothrombin mutation (G20210A; homozygous and heterozygous).

In some embodiments, the patient does not have a history of antiphospholipid antibody syndrome or arterial or venous thromboembolism, atrial fibrillation, significant valvular disease, myocardial infarction, angina, transient ischemic attack, or stroke. Patients who have experienced thrombosis associated with indwelling venous access may be treated with the present methods. In some embodiments, the patient has not had a malignancy within two years, except for basal or squamous cell carcinoma of the skin that has been successfully treated.

In some embodiments, the patient meets one or more of the inclusion criteria and one or more of the exclusion criteria detailed in Example 1 below.

### VI. Treatment Outcomes and Evaluation

In some embodiments, the patient is treated with fitusiran for a period of seven months.

In some embodiments, the present treatment methods reduce the frequency of bleeding episodes in a patient. A bleeding episode is defined as any occurrence of hemorrhage that requires replacement factor or BPA infusion, e.g., hemarthrosis, muscle, or mucosal bleeding. The definition of bleeding episode types described below is based on consensus opinion of International Society on Thrombosis and Haemostasis (ISTH) (Blanchette et al., J Thromb Haemost. (2014) 12(11):1935-9).

The start time of a bleeding episode is defined as the time at which symptoms of a bleeding episode first develop. Bleeding or any symptoms of bleeding at the same location that occurs within 72 hours of the last injection used to treat a bleeding episode at that location is considered a part of the original bleeding event, and will count as one bleeding episode towards the ABR. Any bleeding symptoms that begin more than 72 hours from the last injection used to treat a bleeding episode at that location will constitute a new bleeding event.

In some embodiments, the present treatment methods result in a 50% or greater reduction in the estimated annualized bleed rate (ABR) in the treated population compared to a population treated prophylactically with replacement Factor or BPA (e.g., a 60% or greater reduction). In some embodiments, the observed median ABR in the population treated with fitusiran is reduced to one or less, e.g., to zero. In some embodiments, the historical ABR of the human subject is greater than 1 (i.e., greater than 2, 3, 4, 5, 6, or 7).

A spontaneous bleeding episode refers to a bleeding event that occurs for no apparent or known reason, particularly into the joints, muscles, and soft tissues. A joint bleeding episode is characterized by an unusual sensation in the joint (“aura”) in combination with 1) increasing swelling or warmth over the skin over the joint, 2) increasing pain, or 3) progressive loss of range of motion or difficulty in using the limb as compared with baseline. A muscle bleed may be characterized by pain, swelling and loss of movement over the affected muscle group. A target joint is defined as a joint where three or more spontaneous bleeding episodes in a single joint within a consecutive six-month period has occurred; where there have been less than or equal to two bleeding episodes in the joint within a consecutive 12-month period the joint is no longer considered a target joint. A traumatic bleeding episode is one that is caused by a known injury or trauma. Bleeding episodes sustained during sports and recreation is counted as traumatic bleeding episodes.

In some embodiments, the present treatment methods result in a 40% or greater reduction in the estimated spontaneous annualized bleed rate (AsBR) in the treated population compared to a population treated prophylactically with replacement Factor or BPA (e.g., 50% or greater). In some embodiments, the observed median AsBR in the population treated with fitusiran is reduced to one or less, e.g., zero. In some embodiments, the historical AsBR of the human subject is greater than two.

In some embodiments, the present treatment methods result in a 40% or greater reduction in the estimated annualized joint bleed rate (AJBR) in the treated population compared to a population treated prophylactically with replacement Factor or BPA (e.g., 50% or greater). In some embodiments, the observed median annualized joint bleed rate in the population treated with fitusiran is reduced to one or less, e.g., to zero. In some embodiments, the historical AJBR of the subject is greater than two.

In some embodiments, the present treatment methods result in improved patient-reported outcomes (PROs) as further described below. In some embodiments, the treatment efficacy can be measured by a reduction in the severity of disease as evaluated by the patient based on a valid and reliable hemophilia-specific PRO instrument, for example, the Haemophilia Quality of Life Questionnaire for Adults (“Haem-A-QoL”; von Mackensen et al., Haematologica (2005) 90(s2):115-6, Abstract 0290; Wyrwich et al., Haemophilia (2015) 21(5):578-84). Any positive change resulting in, for example, lessening of severity of disease measured using the appropriate scale, represents adequate treatment using the pharmaceutical compositions as described herein.

Hemophilia, through its associated symptoms, functional limitations and treatment burden, directly impacts the health-related quality of life (HRQoL) of patients. Improving HRQoL is a critical aspect of hemophilia disease management. The present methods improve HRQoL of patients as determined by well-designed, detailed questionnaires. For examples, HRQoL in adult patients (17 years or older, e.g., 18 years or older) can be measured by the Haem-A-QoL questionnaire. In particular embodiments, HRQoL of adult patients may be measured by scores in Haem-A-QoL. See, e.g., von Mackensen et al., Value in Health. (2005) 8(6):A127; von Mackensen et al., J Thrombosis and Haemostasis. (2005) 3 (Supl):P0813; von Mackensen and Gringeri, “Quality of Life in Hemophilia” In: Handbook of Disease Burdens and Quality of Life Measures. Heidelberg: Springer; 2009, pp. 1910-1; and Bullinger et al., Value in Health. (2009) 12(5):808-20; Wyrwich, supra. The Haem-A-QoL questionnaire includes 46 items contributing to 10 domains, including Physical Health (5 items), Feeling (4 items), View of Self (5 items), Sports and Leisure (5 items), Work and School (4 items), Dealing with Hemophilia (3 items), Treatment (8 items), Future (5 items), Family Planning (4 items), and Partnership and Sexuality (3 items).

All Haem-A-QoL items are measured based on a 5-point frequency scale (1=never, 2=rarely, 3=sometimes, 4=often, and 5=all the time). A “Not Applicable” response option is also available for the domains of “Sports & Leisure,” “Work & School,” and “Family Planning” when the question does not apply to the participant. A “Total Score” is also used to represent the average of all 10 domains of the Haem-A-QoL questionnaire. Haem-A-QoL domain scores and the Total Score are transformed to a scale of 0-100 with higher scores representing greater impairment. A decrease in score relative to the corresponding baseline score (score before the treatment being evaluated) indicates an improvement in the patient's quality of life. The questionnaire may be taken before treatment and after treatment with one or more (e.g., two or more, three or more, four or more, five or more, or six or more) doses of fitusiran (e.g., administered subcutaneously at about 50 mg or about 80 mg about once every four weeks or about once a month). For example, the questionnaire may be taken at week 8, 12, 16, 20, 24, 25, 26, or 27 after commencement of fitusiran treatment.

In some embodiments, the present treatment improves the quality of life of patients in one or more of QoL domains (e.g., hemophilia-specific QoL domains). These QoL domains include, for example, domains related to Physical Health, Feeling, View of Self, Sports and Leisure, Work and School, Dealing with Hemophilia, Treatment, Future, Family Planning, and/or Partnership and Sexuality. Improvement in these domains may be evaluated by patient-reported outcome (PRO) and may be aided by questionnaires. For example, improvement in these domains may be reported by a patient to his/her physician, and/or may be scored by a QoL questionnaire.

In some embodiments, the present therapy improves the score in at least one of the Haem-A-QoL domains (e.g., Physical Health, Feeling, View of Self, Sports and Leisure, Work and School, Dealing with Hemophilia, Treatment, Future, Family Planning, and/or Partnership and Sexuality) from baseline, and/or improves the Haem-A-QoL Total Score compared to the score in patients prophylactically treated with replacement factor or BPA. In particular, the present methods may improve the quality of life of hemophilia patients, including improvement (e.g., alleviation and disappearance) of patient-reported hemophilia-related symptoms (e.g., painful swellings and joint pain) and physical functioning (e.g., pain with movement and difficulty walking far) as determined by the Physical Health score and/or the Total Score of Haem-A-QoL.

In some embodiments, the present treatment methods result in an improvement in a patient-reported outcome (PRO) or an improvement in quality of life (QoL). In some embodiments, the improvement is measured using the Haemophilia Quality of Life Questionnaire for Adults (Haem-A-QoL) questionnaire. In some embodiments, the present treatment methods decrease the Haem-A-QoL questionnaire total or physical health domain score, with a decrease of at least 2 units in the fitusiran prophylaxis patient compared to the prophylactically treated BPA or factor patient (e.g., a decrease of at least 3 or at least 4 units).

The EQ-5D-5L is a standardized and disease-generic instrument for use as a measure of QoL outcome. It consists of a questionnaire pertaining to five dimensions (Mobility, Self-Care, Usual Activities, Pain/Discomfort, and Anxiety/Depression). Scoring of the questionnaire is based on five degrees of disability (none, slight, moderate, severe, or extreme). Higher scores indicate better health status. See also e.g., Herdman et al., Qual Life Res (2011) 20(10):1727-36.

The present fitusiran therapy improves the score from at least one of the EQ-5D-5L dimensions (e.g., Mobility, Self-Care, Usual Activities, Pain/Discomfort, and Anxiety/Depression) compared to the prophylactically treated BPA or factor patient. In some embodiments, one or more of the five dimension scores or the total score in the EQ-5D-5L is increased by 0.01 or more units (e.g., 0.02 or more, 0.03 or more, 0.04 or more, 0.05 or more, 0.06 or more, 0.07 or more, 0.08 or more, 0.9 or more, 0.10 or more, 0.11 or more, 0.12 or more, 0.13 or more, 0.14 or more, 0.15 or more, 0.16 or more, 0.17 or more, 0.18 or more, 0.19 or more, or 0.20 or more units).

The present methods improve patient satisfaction with treatment as determined by well-designed, detailed questionnaires, including the Treatment Satisfaction Questionnaire for Medication (TSQM-9). The TSQM-9 is a validated psychometric tool that provides a general measure of patient satisfaction with medication. See also, e.g., Atkinson et al., Health Qual Life Outcomes (2004) 2:12 and Bharmal et al., Health Qual Life Outcomes (2009) 7:36. The TSQM-9 questionnaire includes nine items contributing to three domains, including Effectiveness, Convenience, and Global Satisfaction. TSQM-9 domain scores range from 0 to 100, with higher scores denoting improved HRQoL. An increase in score relative to the corresponding baseline score (score before the treatment being evaluated) indicates an improvement in the patient's satisfaction with treatment. The EQ-5D-5L and TSQM-9 surveys are administered to patients ≥18 years of age.

The present fitusiran therapy improves the score from at least one of the TSQM-9 domains (i.e., Effectiveness, Convenience, and Global Satisfaction) compared to the prophylactically treated BPA or factor patient. In particular, the present methods may improve the treatment satisfaction of hemophilia patients. In some embodiments, one or more of the three domain scores (i.e., Effectiveness, Convenience, and Satisfaction) in TSQM-9 is increased by 1 or more units (e.g., 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, or 20 or more units).

The present fitusiran therapy improves the score from at least one of the HAL or domains (i.e., Lying Down/Sitting/Kneeling/Standing, Functions of the Legs, Functions of the Arms, Use of Transportation, Self Care, Household Tasks, Leisure Activities and Sport, Basic Lower Extremity Activities, Upper Extremity Activities, and Complex Lower Extremity Activities) compared to the prophylactically treated BPA or factor patient. In particular, the present methods may improve the subjective functional ability to perform activities of daily living of hemophilia patients. In some embodiments, one or more of the domain scores in HAL is increased by 1 or more units (e.g., 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, or 20 or more units). In certain embodiments, the present treatment methods result in an improvement indicated by an increase of 0.2 or more units (optionally 0.3 or more, 0.4 or more, 0.5 or more, 0.6 or more, or 0.7 or more units) in the Total Score of HAL.

HRQoL in adolescent patients (12 years or older to 17 years old) can be measured by, e.g., Haemophilia Quality of Life Questionnaire for teenagers (Haemo-QoL) (See, e.g., Bullinger et al., Value Health (2009) 12(5):808-20; and Remor, Int J Behav Med (2013) 20(4):609-17). Haemo-QoL is a shorter version of Haem-A-QoL specifically used for children and adolescents of age groups II/III (8-16 years of age). The Haemo-QoL survey has nine domains, including Physical Health, Feeling, View of Yourself, Dealing with Hemophilia, Treatment, Family, Friends, Others, and Sports. A “Total Score” is also used to represent the average of all nine domains of the Haemo-QoL questionnaire. Scoring for the Haemo-QoL instrument follows the same methodology described above for the Haem-A-QoL instrument. In some embodiments, the present fitusiran therapy reduces one or more of the nine domain scores (e.g., the Physical Health domain score or the Total Score) in Haemo-QoL by 1 or more units (e.g., 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, or 20 or more units).

For all questionnaires described above, the questionnaire may be taken before treatment and after treatment with one or more (e.g., two or more, three or more, four or more, five or more, or six or more) doses of fitusiran (e.g., administered subcutaneously at about 10, about 20, about 50, or about 80 mg about once every four weeks or about once a month or about once every eight weeks or about once every two months). For example, the questionnaire may be taken at week 8, 12, 16, 20, 24, 28, 32, 35, 36, 37, or 38 weeks after commencement of fitusiran treatment.

According to the International Conference on Harmonisation (ICH) E2A guideline Definitions and Standards for Expedited Reporting, and 21 Code of Federal Regulations (CFR) 312.32, IND Safety Reporting, an adverse event (AE) is any untoward medical occurrence in a patient or clinical investigational subject administered a medicinal product and which does not necessarily have a causal relationship with this treatment. Since bleeding episodes are recorded as an efficacy assessment of fitusiran, these will not be treated as AEs unless they meet any of the severe adverse event (SAE) criteria listed below.

An SAE is any untoward medical occurrence that at any dose:
i. results in death,
ii. is life-threatening (an event which places the patient at immediate risk of death from the event as it occurred. It does not include an event that had it occurred in a more severe form might have caused death),
iii. requires in-patient hospitalization or prolongation of existing hospitalization,
iv. results in persistent or significant disability or incapacity,
v. is a congenital anomaly or birth defect, or
vi. is an important medical event that may not be immediately life-threatening or result in death or hospitalization but may jeopardize the patient and may require intervention to prevent one of the other outcomes listed in the definition above (e.g., events include allergic bronchospasm requiring intensive treatment in an emergency room or at home, blood dyscrasias, convulsions, or the development of drug dependency or abuse).

### VII. Articles of Manufacture and Kits

The present invention also provides kits comprising a pharmaceutical composition for use in the present treatment methods. Such kits include one or more vials or one or more pre-filled syringes comprising a pharmaceutical composition of the invention and instructions for use, e.g., instructions for administering a therapeutically effective amount of fitusiran. The kits may optionally further comprise means for administering fitusiran (e.g., an injection device), or means for measuring the inhibition of SERPINCI (e.g., means for measuring the inhibition of SERPINCI mRNA, expression of protein encoded by SERPINCI, and/or SERPINCI activity). Such means for measuring the inhibition of SERPINCI may comprise a means for obtaining a sample from a subject, such as, e.g., a plasma sample. The kits of the invention may optionally further comprise means for determining the therapeutically effective or prophylactically effective amount.

Additional definitions of terminology and exemplary embodiments are described in the Examples and are incorporated by reference herein.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Generally, nomenclature used in connection with, and techniques of hematology, medicine, medicinal and pharmaceutical chemistry, and cell biology described herein are those well-known and commonly used in the art. Further, unless otherwise required by context, singular terms shall include pluralities and plural terms shall include the singular. All publications and other references mentioned herein are incorporated by reference in their entirety. Although a number of documents are cited herein, this citation does not constitute an admission that any of these documents forms part of the common general knowledge in the art. As used herein, the term “approximately” or “about” as applied to one or more values of interest refers to a value that is similar to a stated reference value. In certain aspects, the term refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context.

According to the present disclosure, back-references in the dependent claims are meant as short-hand writing for a direct and unambiguous disclosure of each and every combination of claims that is indicated by the back-reference. Further, headers herein are created for ease of organization and are not intended to limit the scope of the claimed invention in any manner.

In order that this invention may be better understood, the following examples are set forth. These examples are for purposes of illustration only and are not to be construed as limiting the scope of the invention in any manner.

List of Abbreviations and Definitions of Terms

| Abbreviation or | |
|---|---|
| Specialist Term | Explanation |
| ABR | Annualized bleeding rate |
| ADA | Antidrug antibody |
| AE | Adverse event |
| ALP | Alkaline phosphatase |
| ALT | Alanine aminotransferase |
| ANCOVA | Analysis of covariance |
| Anti-HCV Ab | Anti-hepatitis C antibody |
| aPCC | Activated prothrombin complex concentrates |
| AST | Aspartate aminotransferase |
| AT | Antithrombin |
| AUC | Area under the concentration-time curve |
| BPA | Bypassing agent |
| BU | Bethesda units |
| BUN | Blood urea nitrogen |
| CBC | Complete blood count |
| CFR | Code of Federal Regulation |
| CI | Confidence interval |
| CL/F | Apparent clearance |
| Cₘₐₓ | Maximum plasma concentration |
| CRF | Case report form |
| CRP | C-reactive protein |
| CT | Computed tomography |
| CVST | Cerebral venous sinus thrombosis |
| CYP P450 | Cytochrome P450 |
| DMC | Data Monitoring Committee |
| DTP | Duties and taxes paid |
| ECG | Electrocardiogram |
| eCRF | Electronic case report form |
| eDiary | Electronic diary |
| EE | Efficacy-evaluable |
| EQ-5D | EuroQol-5 dimension |
| ET | Early termination |
| FIX | Factor IX |
| FV | Factor V |
| FVII | Factor VII |
| FVIII | Factor VIII |
| FX | Factor X |
| GalNAc | N-Acetylgalactosamine |
| GCP | Good Clinical Practice |
| GGT | Gamma glutamyl transferase |
| GLP | Good Laboratory Practice |
| Haem-A-QOL | Hemophilia Quality of Life Questionnaire for Adults |
| Haemo-QOL | Hemophilia Quality of Life Questionnaire for Children |
| | and Adolescents |
| HAL | Hemophilia Activities List |
| HBc Ab | Hepatitis B core antibody |
| HBs Ag | Hepatitis B surface antigen |
| HIV | Human immunodeficiency virus |
| HRQOL | Health-related quality of life |
| IB | Investigator's Brochure |
| ICF | Informed Consent Form |
| ICH | International Conference on Harmonisation |
| IEC | Independent Ethics Committee |
| IMP | Investigational medicinal product |
| INR | International normalized ratio |
| IP | Investigational product |
| IRB | Institutional Review Board |
| IRS | Interactive response system |
| ISR | Injection site reaction |
| ISTH | International Society on Thrombosis and Haemostasis |
| ITI | Immune tolerance induction |
| ITT | Intent-to-treat |
| IV | Intravenous |
| LFT | Liver function test |
| MAD | Multiple ascending dose |
| MDRD | Modification of Diet in Renal Disease |
| MMRM | Mixed effect repeated measures |
| mRNA | Messenger ribonucleic acid |
| NHP | Non-human primate |
| NOAEL | No observed adverse effect level |
| PCC | Prothrombin complex concentrate |
| PD | Pharmacodynamics |
| pedHAL | Pediatric HAL |
| PK | Pharmacokinetics |
| PP | Per-protocol |
| PT | Prothrombin time |
| QOL | Quality of life |
| RNAi | Ribonucleic acid interference |
| rVIIa | Recombinant factor VIIa |
| SAD | Single ascending dose |
| SAE | Serious adverse event |
| SAP | Statistical Analysis Plan |
| SC | Subcutaneous |
| SD | Standard deviation |
| SDay | Surgery day |
| siRNA | Small interfering ribonucleic acid |
| SSC | Scientific Standardization Committee |
| SUSAR | Suspected unexpected serious adverse reactions |
| t_{1/2β} | Elimination half-life |
| tₘₐₓ | Time to maximum plasma concentration |
| TG | Thrombin generation |
| TSQM | Treatment Satisfaction Questionnaire for Medication |
| ULN | Upper limit of normal |
| V/F | Apparent volume of distribution |

### EXAMPLES

### Example 1: Clinical Trial Protocol for an Open-Label, Switching Study to Describe the Efficacy and Safety of Fitusiran Prophylaxis in Patients with Hemophilia A and B Previously Receiving Factor or Bypassing Agent Prophylaxis

The Example describes an open-label, Phase 3 switching study designed to demonstrate the efficacy and safety of fitusiran in patients with hemophilia A or B, who are currently treated with prophylactic regimens of factor concentrates or BPAs. The switching design allows for an intra-patient control to enable examination of the effect of the two treatment methods through comparison of the median ABR during the factor or BPA prophylaxis period and the median ABR of the same patient group when receiving fitusiran, while limiting confounding effects of different patient bleeding phenotypes and prophylaxis therapy variability. Inhibitor patients with hemophilia B may have a high unmet need despite prophylactic BPA therapy, with limited other treatment options. Therefore, a limited number of inhibitor patients with hemophilia B who are not adequately responding to prophylactic BPA therapy could enroll directly into the fitusiran treatment period, thereby skipping the 6-month BPA prophylaxis period. The onset period duration reflects modeling data that estimates it takes approximately 28 days to reach the therapeutic target range in the majority of patients. Given that the study design employed is a single treatment arm, with a switch from prophylaxis to fitusiran for each patient, the study is not blinded.

The primary endpoint of the study is ABR in the fitusiran efficacy period and the factor or BPA prophylaxis period. ABR is a well-established endpoint that has been used as the primary endpoint in global approvals of factor replacement and BPA products. Secondary endpoints characterize annualized spontaneous and joint bleeding rates, change in Haem-A-QoL physical health score and total score in patients 17 years of age, ABR in the onset period, overall safety profile and the consumption of factor/BPA.

Characterization of bleeding episodes is clinically relevant to assess overall bleeding episode protection. Joint bleeding episodes result in pain and hemarthrosis, leading to progressive joint destruction, and hence are important to assess. The Haem-A-QOL is a hemophilia-specific HRQOL survey instrument, has been used in other hemophilia clinical trials, has been validated, reviewed by clinicians, and is considered the most appropriate HRQOL tool available for use in the study.

The study population will be comprised of males ≥12 years of age; it is appropriate to study fitusiran in adolescents (patients ≥12 to <18 years of age) because the pathophysiology of disease progression and bleeding episode management is the same as adults and self-management of hemophilia typically begins at 12 years of age.

In the event of a breakthrough bleeding episode, on-demand use of factors or BPAs will be permitted throughout the entire study duration.

Duration of Treatment

The duration of treatment with fitusiran is 7 months. The estimated total time on the study, inclusive of screening, for each patient is up to 15 months for all patients who enroll in the extension study, except for patients in the subgroup of Cohort A, which is up to 9 months. The estimated total time on study may be up to 21 months (up to 15 months for patients in the subgroup of Cohort A) for patients who do not enroll in the extension study due to the requirement for an additional six months of follow-up for monitoring of AT levels.

Study Objectives and Endpoints

The primary objective of this study is to characterize the frequency of bleeding episodes while receiving fitusiran treatment, relative to the frequency of bleeding episodes while receiving factor or bypassing agent (BPA) prophylaxis.

The secondary objectives of this study are:
to characterize the following while receiving fitusiran treatment, relative to receiving factor or BPA prophylaxis:
   the frequency of spontaneous bleeding episodes,
   the frequency of joint bleeding episodes, and
   health related quality of life (HRQOL) in patients ≥17 years of age;
to characterize the frequency of bleeding episodes during the onset and treatment periods in patients receiving fitusiran;
to characterize the safety and tolerability of fitusiran; and
to characterize the annualized weight-adjusted consumption of factor/BPA while receiving fitusiran treatment, relative to receiving factor or BPA prophylaxis.

The exploratory objectives are:
to characterize the effects of fitusiran on the following patient-reported outcomes while receiving fitusiran treatment, relative to receiving factor or BPA prophylaxis:
   patient satisfaction with fitusiran,
   patient activity, and
   HRQOL in adolescents (≥12 to <17 years of age);
to characterize the pharmacodynamic (PD) effect, pharmacokinetics (PK), and immunogenicity of fitusiran;
to characterize the effects of fitusiran on joint status while receiving fitusiran treatment, relative to receiving factor or BPA prophylaxis; and
to characterize the effects of fitusiran on patient resource use, relative to receiving factor or BPA prophylaxis.

The primary endpoint of this study is to evaluate the Annualized Bleeding Rate (ABR) in the fitusiran efficacy period and the factor or BPA prophylaxis period.

The secondary endpoints are to evaluate:
annualized spontaneous bleeding rate in the fitusiran efficacy period and the factor or BPA prophylaxis period;
annualized joint bleeding rate (AJBR) in the fitusiran efficacy period and the factor or BPA prophylaxis period; and
change in Haem-A-QOL physical health score and total score in the fitusiran treatment period and the factor or BPA prophylaxis period.

The exploratory endpoints are:
Change in the following in the fitusiran treatment period:
   treatment Satisfaction Questionnaire for Medication (TSQM) domain scores,
   Haemophilia Activities List (HAL) score,
   paediatric HAL (pedHAL) score,
   euroQol-5 Dimensions (EQ-5D) score,
   Haemo-QOL score, and/or
   Hemophilia Joint Health Score (HJHS);
number of target joint bleeding episodes;
incidence and titer of antidrug antibodies to fitusiran in the fitusiran treatment period;
antithrombin (AT) activity level over time;
thrombin generation over time;
fitusiran plasma levels; and
change in patient resource use (e.g., work/school attendance, visits to doctor/hospital).

The safety endpoint was to evaluate the incidence, severity, seriousness, and relatedness of adverse events.

Study Design

The study evaluates male patients, aged ≥12 years, with hemophilia A or B, who have switched from prior bypassing agent (BPA, Cohort A) or factor (Cohort B) prophylaxis. Cohort A patients have inhibitory antibodies to Factor VIII or Factor IX, whereas Cohort B patients do not have inhibitor antibodies to Factor VIII or Factor IX.

A subgroup of Cohort A patients includes hemophilia B patients with inhibitory antibodies to Factor IX who are not responding adequately to BPA prophylaxis treatment (historical ABR≥20).

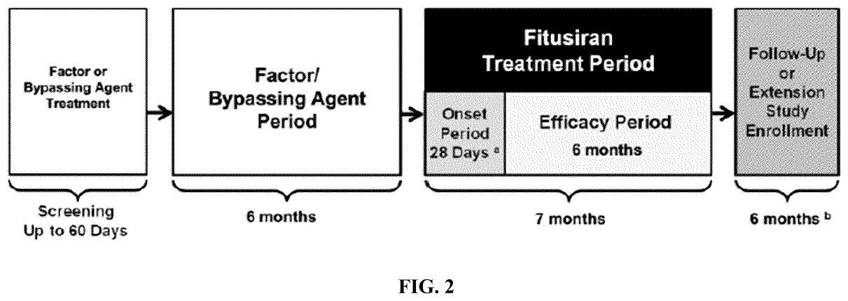

The study has three periods defined by type of prophylaxis regimen (FIG. 2):
Six-month factor or BPA prophylaxis period in which patients continued their pre-study, regularly scheduled prophylaxis regimen with factors or BPAs,
1-month onset period in which patients receive first dose of fitusiran while continuing their factor or BPA prophylaxis for up to 7 days, and
6-month fitusiran efficacy period in which patients receive fitusiran as prophylaxis.

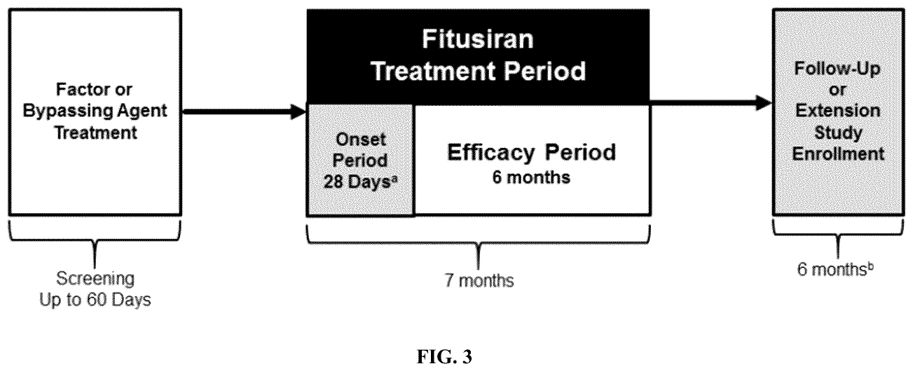

The subgroup of Cohort A patients who are not responding adequately to BPA prophylaxis treatment will not participate in the six-month BPA prophylaxis period and will start to directly receive fitusiran (in the one-month onset period) after the screening period (FIG. 3).

Together, the one-month onset period and the six-month fitusiran efficacy period constitute the fitusiran treatment period.

On-demand use of factor concentrates or BPAs is defined as the use of these agents, as needed, for episodic bleeding, and not on a regular regimen intended to prevent spontaneous bleeding. Throughout the study, patients in the fitusiran treatment period received on-demand treatment for breakthrough bleeding episodes with factors or BPAs, as appropriate. For patients in the fitusiran treatment period who have received at least 1 dose of fitusiran and are being treated for breakthrough bleeding episodes, it is recommended to follow the guidelines provided in Table 3.

Following the screening and prophylaxis periods (or following the screening period for the subgroup of Cohort A enrolling directly into the fitusiran treatment period) all patients were treated with fitusiran for a total of seven months. Therefore, the overall fitusiran treatment period was defined as the onset period (day 1-28 after receipt of the first dose, during which the AT lowering capacity of fitusiran is increasing but has not yet reached therapeutic levels) plus the efficacy period (day 29 and after, when the AT lowering capacity of fitusiran has achieved therapeutic target range).

Study Population

This study will include males with severe hemophilia A or B with or without inhibitors, aged ≥12 years, who have been prescribed prophylactic treatment with factor concentrates or BPAs for at least 6 months prior to screening. Diagnosis of severe hemophilia A or B will be based on a central laboratory measurement or documented medical record evidence of FVIII level <1% or FIX level ≤2%. Patients with inhibitors must have used BPAs for prophylaxis for at least the last 6 months prior to screening and must meet one of the following Nijmegen-modified Bethesda assay results criteria: 1) inhibitor titer of ≥0.6 BU/mL at screening; 2) inhibitor titer of <0.6 BU/mL at screening with medical record evidence of 2 consecutive titers ≥0.6 BU/mL; or 3) inhibitor titer of <0.6 BU/mL at screening with medical record evidence of anamnestic response.

The subgroup of patients in Cohort A patients must additionally meet the following criteria to be eligible to start treatment with fitusiran directly after the screening period: 1) hemophilia B with inhibitory antibody to Factor IX as defined above; 2) Not responding adequately to BPA treatment (historical ABR ≥20) prior to enrollment; and 3) 6-month BPA prophylaxis period should be omitted as deemed appropriate. A minimum of 2 bleeding episodes requiring BPA treatment within the last 6 months prior to screening is required.

Patients without inhibitors must have used factor concentrates for prophylaxis for at least the last 6 months prior to Screening and must meet each of the following criteria: 1) Nijmegen-modified Bethesda assay inhibitor titer of <0.6 BU/mL at screening; 2) no use of bypassing agents to treat bleeding episodes for at least the last 6 months prior to screening; and 3) no history of immune tolerance induction therapy within the last 3 years prior to Screening. A minimum of 1 bleeding episode requiring factor treatment within the last 12 months prior to Screening is required.

The inclusion criteria are further described as follows:
I 01. males ≥12 years of age;
I 02. severe hemophilia A or B, as evidenced by a measurement at screening or documented medical record evidence of FVIII<1% or FIX level ≤2%;
I 03. a minimum of two bleeding episodes requiring BPA treatment within the last six months prior to screening for patients with inhibitory antibodies to Factor VIII or Factor IX (Cohort A); or a minimum of one bleeding episode requiring factor treatment within the last 12 months prior to screening for patients without inhibitory antibodies to Factor VIII or Factor IX (Cohort B);
I 04. must meet either the definition of inhibitor or non-inhibitor patient as below:
   Inhibitor (Cohort A): use of BPAs for prophylaxis and for any bleeding episodes for at least the last six months prior to Screening, and meet one of the following Nijmegen-modified Bethesda assay results criteria:
      inhibitor titer of ≥0.6 BU/mL at Screening, or
      inhibitor titer of <0.6 BU/mL at Screening with medical record evidence of 2 consecutive titers ≥0.6 BU/mL, or
      inhibitor titer of <0.6 BU/mL at Screening with medical record evidence of anamnestic response;
      the subgroup of Cohort A patients must additionally meet the following criteria to be eligible to start treatment with fitusiran directly after the screening period:
         hemophilia B with inhibitory antibody to Factor IX as defined above, and
         not responding adequately to BPA treatment (historical ABR 20) prior to treatment;
   non-inhibitor: use of factor concentrates for prophylaxis and for any bleeding episodes for at least the last six months prior to Screening, and meet each of the following criterion:
      Nijmegen-modified Bethesda assay inhibitor titer of <0.6 BU/mL at Screening,
      no use of bypassing agents to treat bleeding episodes for at least the last 6 months prior to Screening and,
      no history of immune tolerance induction therapy within the past three years prior to Screening; and
I 05. prescribed (and adhered to) prophylactic treatment (documented in the medical or pharmacy records) of hemophilia with factor concentrates or BPAs for at least six months prior to Screening; the regimen must be consistent with the approved prescribing information for the product or local recommendations, allowing for adjustment to individual patient response, and designed to decrease spontaneous bleeding; and
I 06. Adherent to the prescribed prophylactic therapy for at least 6 months prior to Screening per Investigator assessment.

The exclusion criteria are further described as follows:
E 01. known co-existing bleeding disorders other than hemophilia A or B, i.e, Von Willebrand's disease, additional factor deficiencies, or platelet disorders;
E 02. current participation in immune tolerance induction therapy (ITI);
E 03. AT activity <60% at Screening, as determined by central laboratory measurement.
E 04. presence of clinically significant liver disease, or as indicated by any of the conditions below:
   INR>1.2;
   ALT and/or AST>1.5×upper limit of normal reference range (ULN);
   Total bilirubin >ULN (>1.5×ULN in patients with Gilbert's Syndrome);
   History of portal hypertension, esophageal varices, or hepatic encephalopathy; or
   presence of ascites by physical exam;
E 05. Hepatitis C virus antibody positive, except patients with a history of HCV infection who meet both of the following conditions:
   completed curative treatment at least 12 weeks prior to enrollment and attained sustained virologic response as documented by a negative HCV RNA at screening, or they have spontaneously cleared infection as documented by negative HCV RNA at Screening.
   no evidence of cirrhosis according to one of the following assessments:
      FibroScan <12.5 kPa (where available), or
      FibroTest score <0.75 and APRI <2 (if FibroScan unavailable) E 06.
E 06. presence of acute hepatitis, i.e., hepatitis A, hepatitis E;
E 07. presence of acute or chronic hepatitis B infection (IgM anti-HBc antibody positive or HBsAg positive);
E 08. platelet count ≤100,000/μL;
E 09. presence of acute infection at Screening;
E 010. known to be HIV positive with CD4 count <200 cells/μL;
E 011. inadequate renal function, as evidenced by estimated glomerular filtration rate <45 mL/min/1.73 m2 (using the Modification of Diet in Renal Disease [MDRD] formula);
E 012. co-existing thrombophilic disorder, as determined by presence of any of the below as identified at central laboratory (or via historical results, where available):
   FV Leiden mutation (homozygous or heterozygous),
   Protein S deficiency, or
   Protein C deficiency;
   prothrombin mutation (G20210A; homozygous and heterozygous);
E 013. history of antiphospholipid antibody syndrome;
E 014. history of arterial or venous thromboembolism, atrial fibrillation, significant valvular disease, myocardial infarction, angina, transient ischemic attack, or stroke; Patients who have experienced thrombosis associated with indwelling venous access may be enrolled; or
E 015. had a malignancy within two years, except for basal or squamous cell carcinoma of the skin that has been successfully treated.

Dosing Regimens and Formulation

Fitusiran solution for injection (SC use) is supplied as a sterile solution. Patients receive 80 mg fitusiran as an SC injection once monthly (or 50 mg fitusiran every two months), for a total of seven months.

Antithrombin Level Criteria for a Dose Adjustment

Upon the first AT level <15%, the patient in this study has another AT activity level sample drawn within 1 week of site receipt of the result. If this result is <15%, this will be considered the second AT activity level <15%. In this study, patients receiving fitusiran at a dose of 50 mg Q2M with more than 1 AT activity level <15% at any time during the study discontinue fitusiran.

Routine Use of Factor or Bypassing Agent Prophylaxis in the Factor or Bypassing Agent Prophylaxis Period

During the factor or BPA prophylaxis period, patients will continue to receive prophylaxis with their usual products on a regimen consistent with recommendations in the approved prescribing information, allowing for adjustment to individual patient response, and designed to decrease spontaneous bleeding. The regimen used during the factor or BPA prophylaxis period must have a minimum frequency of administration as presented in Table 2 (supra).

The subgroup of Cohort A patients who are not responding adequately to BPA prophylaxis treatment will not participate in this BPA prophylaxis period and will directly start the fitusiran treatment period after the screening period.

Management of Factor or Bypassing Agent Prophylaxis During the Transition to the Fitusiran Treatment Period

The first 28 days of fitusiran treatment is referred to as the onset period. During the onset period AT lowering will be progressing toward therapeutic levels. Patients will continue factor or BPA prophylaxis with minimum frequency as in Table 2, for the first 7 days of the fitusiran onset period. Subsequent to Day 7 of the fitusiran treatment period, factor concentrates or BPAs should be administered only for bleeding episodes or if needed in advance of invasive medical procedures.

Bleeding Episode Management Recommendations for Patients During the Fitusiran Treatment Period

See Section IV of the detailed description above.

Study Assessments

A bleeding episode is defined as any occurrence of hemorrhage that requires BPA or replacement factor infusion, e.g., hemarthrosis, muscle, or mucosal bleeding. The definition of bleeding episode types described below is based on consensus opinion of International Society on Thrombosis and Haemostasis (ISTH) (Blanchette et al., J Thromb Haemost. (2014) 12(11):1935-9).

The start time of a bleeding episode was considered the time at which symptoms of a bleeding episode first develop. Bleeding or any symptoms of bleeding at the same location that occurs within 72 hours of the last injection used to treat a bleeding episode at that location was considered a part of the original bleeding event, and will count as one bleeding episode towards the ABR. Any bleeding symptoms that begin more than 72 hours from the last injection used to treat a bleeding episode at that location will constitute a new bleeding event.

A spontaneous bleeding episode is a bleeding event that occurs for no apparent or known reason, particularly into the joints, muscles, and soft tissues.

A joint bleeding episode is characterized by an unusual sensation in the joint (“aura”) in combination with 1) increasing swelling or warmth over the skin over the joint, 2) increasing pain, or 3) progressive loss of range of motion or difficulty in using the limb as compared with baseline.

A muscle bleed may be characterized by pain, swelling and loss of movement over the affected muscle group.

A target joint is defined as a joint where three or more spontaneous bleeding episodes in a single joint within a consecutive six-month period has occurred; where there have been less than or equal to two bleeding episodes in the joint within a consecutive 12-month period the joint is no longer considered a target joint.

A traumatic bleeding episode is one that is caused by a known injury or trauma. Bleeding episodes sustained during sports and recreation was counted as traumatic bleeding episodes.

Patient-reported outcomes are utilized to assess health-related quality of life (HRQOL), physical activity, and treatment satisfaction and health utility. The Hemophilia Quality of Life Questionnaire for adults (Haem-A-QOL) is a psychometrically tested QOL assessment instruments for patients with hemophilia. The Haem-A-QOL is provided to patients ≥17 years of age, and includes 46 items contributing to 10 QOL domains (physical health, feelings, view of yourself, sports and leisure, work and school, dealing with hemophilia, treatment, future, family planning, partnership and sexuality). Scoring for each item is based on a 5-point Likert scale (never, rarely, sometimes, often, and all the time), and higher scores represent greater impairment.

Antithrombin Activity

AT activity level will be assessed twice a month (about every two weeks) for the first two months and then monthly (about every four weeks) thereafter. On dosing days, samples will be collected within 4 hours prior to dosing. Antithrombin protein may be measured in a subset of plasma samples for correlation.

Statistical Methodology

The primary analysis will be performed on the EAS and will include all bleeding episodes occurring in the factor or BPA prophylaxis period (Day −162 to Day −1) and the fitusiran efficacy period (Day 29 to Day 190). If a patient does not have bleeding episode data collected after Day 28 (e.g., due to early study discontinuation), the available bleeding episode data starting from Day 1 will be used in the primary analysis. To avoid confounding the treatment effect, bleeding episode data during and after major surgery, antithrombin administration, major trauma, or initiation of prophylaxis treatment with factors or BPAs during the fitusiran treatment period will be excluded from the primary analysis.

The number of bleeding episodes will be analyzed using a repeated measures negative binomial model with fixed effect of treatment period. The logarithm number of days that each patient spends in the efficacy period matching the bleeding episode data being analyzed will be included as an offset variable to account for unequal follow-up time due to early withdrawal or surgery. The ratio of bleeding rates in the fitusiran efficacy period to the factor or BPA prophylaxis period and its 95% CI and p-value will be presented.

In addition, as a contrast Bayesian analyses will be performed to summarize the point estimates of the posterior probability of a clinically significant treatment effect, along with associated measures of uncertainty. The estimated mean ABRs in these 2 periods along with their 95% CIs will be presented from this model. In addition, summary statistics for ABR, including the median and interquartile range, will be presented for each treatment arm, where ABR is defined as:

(total number of qualifying bleeding episodes/total number of days in the respective period)×365.25

Spontaneous bleeding episodes and joint bleeding episodes will be analyzed using the same method as primary analysis of ABR. Summary statistics, including the median and interquartile range, for annualized spontaneous bleeding rate and annualized joint bleeding rate will be reported.

Change in Haem-A-QOL physical health score and total score (in patients 17 years of age) in the factor or BPA prophylaxis period and fitusiran treatment period will be summarized descriptively. A mixed model for repeated measures analysis may be performed as deemed appropriate.

The bleeding episodes in the fitusiran onset period and in the fitusiran treatment period will be analyzed using a negative binomial model with logarithm of follow-up time in the period as an offset parameter. Summary statistics, including the median and interquartile range, for the ABR in 2 periods will be reported.

The secondary endpoint of annualized weight-adjusted consumption of factor/BPA injections will be summarized using descriptive statistics.

The fitusiran efficacy period (fitusiran prophylaxis) was defined as starting on Day 29 after the first dose of fitusiran up to Day 190, or the last day of bleeding follow up, whichever is the earliest. The factor/BPA prophylaxis period was defined as starting on Day −168 to Day −1, or the last day of bleeding follow up, whichever is the earliest. The factor/BPA prophylaxis period is defined as starting on day −168 to day −1, or the last day of bleeding follow up, whichever is the earliest.

### Example 2: Phase 3 Clinical Trial Results

Clinical trial results were obtained in accordance with the trial protocol described in Example 1. The patients were males aged 12 years with hemophilia A or B, with or without inhibitors, who had prior factor/BPA prophylaxis. Participants continued factor/BPA prophylaxis (6 months) before switching to once-monthly 80 mg SC fitusiran prophylaxis (7 months). Primary endpoint was ABR in the factor/BPA prophylaxis period (Day−168 to Day−1) and fitusiran efficacy period (Day 29 to Day 190). Secondary endpoints included spontaneous ABR (AsBR), joint ABR (AjBR), and health-related quality of life (HRQoL). Safety and tolerability were assessed.

Results are presented for Safety Analysis Set 1 (SAS1) and Efficacy Analysis Set 1 (EAS1) which include participants who enrolled and then received 80 mg fitusiran QM. The efficacy Analysis Set 1 included participants who received factor or BPA prophylaxis and at least one dose of fitusiran.

Participant Disposition

Of 99 participants screened, 80 (normalized to 100%) were enrolled. 30 participants had inhibitory antibodies to Factor VIII or Factor IX (Cohort A [inhibitor]) and 50 participants did not have inhibitory antibodies to Factor VIII or Factor IX (Cohort B [non-inhibitor]). 78 (97.5%) of 80 participants enrolled entered the factor/BPA prophylaxis period and 67 (83.8%) completed the factor/BPA prophylaxis period. Two (2.5%) of 80 participants enrolled started directly with fitusiran 80 mg QM (subgroup of Cohort A) (Table 4).

TABLE 4
Factor/BPA Prophylaxis: Day −168 to Day −1

| | Cohort A: | Cohort B: |
|---|---|---|
| | Inhibitor | Non-inhibitor |
| Started (Enrolled) | 30 | 50 |
| Started Directly with Fitusiran | 2 | 0 |
| Started with Factor or BPA Prophylaxis | 28 | 50 |
| Completed | 23 | 46 |
| Not Completed | 7 | 4 |
| Withdrawal by Subject | 0 | 2 |
| Adverse Event | 1 | 0 |
| Parent withdrew consent | 1 | 0 |
| Switched to treatment not per protocol | 4 | 0 |
| Related to Coronavirus Disease 2019 | 1 | 0 |
| Did not meet trial eligibility | 0 | 2 |

Of the 67 participants that completed the factor/BPA prophylaxis period, 65 started with fitusiran 80 mg QM, and two started with fitusiran 50 mg Q2M. Of the 67 (83.8%) participants that started fitusiran 80 mg QM, 13 (16.3%) discontinued and 54 (67.5%) completed fitusiran treatment. Nine (11.3%) participants discontinued fitusiran 80 mg QM due to voluntary dosing pause per sponsor decision and completed the study. Two (2.5%) participants discontinued fitusiran due to AEs. Of the two participants treated with fitusiran 50 mg Q2M, one discontinued fitusiran due to more than one AT value <15% and completed the study, and one withdrew consent. 64 (80.0%) participants completed the study (Tables 5 and 6).

TABLE 5
Fitusiran Treatment: Day 1 to Day 190

| | Cohort A: | Cohort B: |
|---|---|---|
| | Inhibitor | Non-inhibitor |
| Started | 23 | 46 |
| Received 80 mg QM & Part of Safety | 21 | 46 |
| Analysis Set 1 (SAS 1) | | |
| Received 50 mg Q2M & Part of Safety | 2¹ | 0 |
| Analysis Set 2 (SAS 2) | | |
| Efficacy Analysis Set 1 (EAS 1) | 19 | 46 |
| Efficacy Analysis Set 2 (EAS 2) | 2² | 0 |
| Completed | 17 | 37 |
| Not Completed | 6 | 9 |
| Withdrawal by Subject | 1 | 0 |
| More than 1 antithrombin (AT) measurement | 1 | 0 |
| less than 15% | | |
| Study drug on hold | 4 | 5 |
| Participant's decision | 0 | 2 |
| Adverse Event | 0 | 2 |

¹Participants received fitusiran 50 mg Q2M dosing regimen after dose pause and amended protocol 5, dated 25 Nov. 2020. They were considered as safety analysis set 2 (SAS 2) and were subjected only to safety analysis and not to main efficacy analysis (Efficacy Set 1).
²Efficacy Analysis Set 2 (EAS 2) included all participants in the SAS 2 who received BPA prophylaxis and fitusiran 50 mg Q2M after dose pause, protocol amendment 5 (dated 25 Nov. 2020) and dose resumption.

TABLE 6
Baseline Measures

| | | Cohort A: | | Cohort B: | | | |
|---|---|---|---|---|---|---|---|
| | | Inhibitor | | Non-Inhibitor | | Total | |
| Overall Number of Participants | | 23 | | 46 | | 69 | |
| Age, Continuous | Number of | 23 | | 46 | | 69 | |
| Mean, Years | Participants | | | | | | |
| (Standard | Analyzed | | | | | | |
| Deviation) | Age | 27.7 | (15.9) | 23.5 | (7.3) | 24.9 | (11.0) |
| Sex: | Number of | 23 | | 46 | | 69 | |
| Female, | Participants | | | | | | |
| Male | Analyzed | | | | | | |
| Measurement | Female | 0 | | 0 | | 0 | |
| Type: Count of | Male | 23 | (100%) | 46 | (100%) | 69 | (100%) |
| Participants | | | | | | | |
| Unit of Measure: | | | | | | | |
| Participants | | | | | | | |
| Race | Number of | 23 | | 46 | | 69 | |
| (NIH/OMB) | Participants | | | | | | |
| Measure Type: | Analyzed | | | | | | |
| Count of | American Indian | 0 | (0%) | 0 | (0%) | 0 | (0%) |
| Participants | or Alaska Native | | | | | | |
| Unit of Measure: | Asian | 4 | (17.39%) | 17 | (36.96%) | 21 | (30.43%) |
| Participants | Native Hawaiian | 0 | (0%) | 0 | (0%) | 0 | (0%) |
| | or Other Pacific | | | | | | |
| | Islander | | | | | | |
| | Black or African | 1 | (4.35%) | 0 | (0%) | 1 | (1.45%) |
| | American | | | | | | |
| | White | 18 | (78.26%) | 27 | (58.7%) | 45 | (65.22%) |
| | More than one | 0 | (0%) | 0 | (0%) | 0 | (0%) |
| | race | | | | | | |
| | Unknown or not | 0 | (0%) | 2 | (4.35%) | 2 | (2.9%) |
| | reported | | | | | | |

Population Characteristics

Among the 80 participants enrolled, 57 were participants with hemophilia A (21 in Cohort A [inhibitor] and 36 in Cohort B [non-inhibitor]) and 23 were participants with hemophilia B (9 in Cohort A [inhibitor] and 14 in Cohort B [non-inhibitor]). All participants were male and the median age was 23.0 years. 19 (29.2%) patients were adolescents (12-17 years), 45 (69.2%) were adults (18-64 years), and one participant was 65 years or older.

A total of 42 (64.6%) participants were White, 20 (30.8%) were Asian, two (3.1%) were “Other,” and one participant was Black or African American. The median body mass index was 23.6 kg/m².

For participants in Cohort A (inhibitor), the median (IQR) number of bleeding episodes in the six months prior to screening was 4.0 (2.0 to 6.0); for participants in Cohort B (non-inhibitor), the median number of bleeding episodes in the 12 months prior to screening was 2.0 (1.0 to 4.0). The majority of participants (18 [94.7%]) in Cohort A (inhibitor) had a highest historical inhibitory antibody titer ≥5 BU/ml. The baseline demographics and characteristic of the patients were generally similar between the cohorts, and are summarized in Table 7.

TABLE 7
Baseline Demographics and Characteristics
of Patients in Cohorts A and B

| | Participants with | | Participants without | |
|---|---|---|---|---|
| | inhibitors | | inhibitors | |
| Characteristics | (Cohort A; n = 19) | | (Cohort B; n = 46) | |
| Age, mean (SD), years | 27.8 | (17.1) | 23.5 | (7.3) |
| Weight, mean (SD), kg | 68.2 | (16.2) | 71.7 | (15.3) |
| Hemophilia A | 14 | (73.7) | 36 | (78.3) |
| Hemophilia B | 5 | (26.3) | 10 | (21.7) |
| Median number of bleeding | 4.0 | | N/A | |
| episodes in the last six | | | | |
| months prior to screening | | | | |
| Median number of bleeding | NA | | 2.0 | |
| episodes in the 12 months | | | | |
| prior to screening | | | | |
| Number of target joints at | 0.0 | (0.0; 1.0) | 0.0 | (0.0; 0.0) |
| baseline, | | | | |
| median (Q1, Q3) | | | | |

Efficacy Results

A total of 80 patients were enrolled and 65 were evaluable for efficacy analyses (inhibitor/non-inhibitor, n=19/46; hemophilia A/hemophilia B, n=50/15). The mean (SD) age was 24.8 (11.2) years.

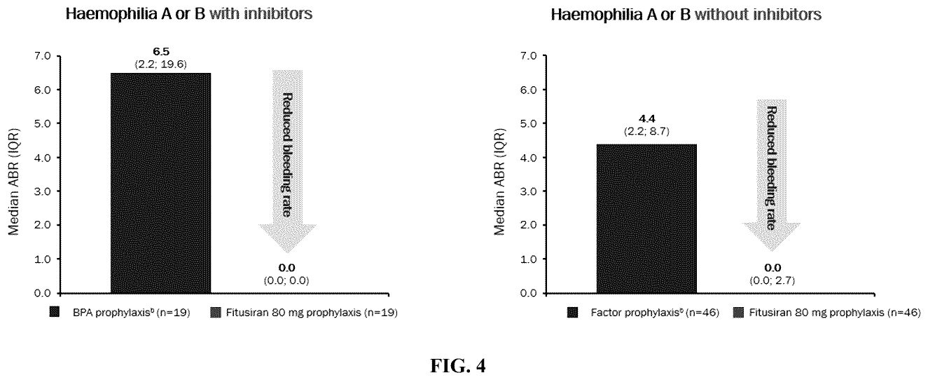

Overall, for participants treated with 80 mg QM fitusiran prophylaxis, the estimated ABR was 2.908 (95% confidence interval [CI], 1.727 to 4.898) during the efficacy period, and 7.482 (95% CI, 5.520 to 10.141) during the factor or BPA prophylaxis period, representing a statistically significant reduction of 61.1% (95% CI, 32.5% to 77.6%) in treated bleeds in favor of fitusiran prophylaxis (P=0.0008). Overall, median (IQR) observed ABRs were 0.00 (0.00; 2.25) and 4.35 (2.17; 10.87) during the fitusiran efficacy period and the factor or BPA prophylaxis period, respectively (Table 8). 63.1% of participants experienced zero treated bleeds during the fitusiran efficacy period compared to 16.9% during the factor or BPA period (Table 9). In participants with inhibitors (Cohort A) fitusiran was associated with low median (IQR) ABR of 0.00 (0.00; 0.00) and a significant 79.7% (95% CI, 43.8% to 92.6%) ABR reduction compared to BPA prophylaxis (P=0.0021). In participants without inhibitors (Cohort B) fitusiran was associated with low median (IQR) ABR of 0.00 (0.00; 2.65) and a clinically meaningful 46.4% ABR reduction (numerical) compared to factor prophylaxis (P=0.0598) (FIG. 4). Fitusiran prophylaxis resulted in a significant 61.1% reduction in bleeding rate (p=0.0008) compared to factor/BPA prophylaxis.

Fitusiran achieved the study primary endpoint and significantly reduced the frequency of bleeding episodes; observed median (IQR) ABR for treated bleeds was 0.0 (0.0; 2.3) in the fitusiran efficacy period and 4.35 (2.2; 10.9) in the factor/BPA prophylaxis period. 41 (63.1%) participants treated prophylactically with fitusiran experienced zero treated bleeds.

TABLE 8
Estimated ABR and Rate Ratio in the Factor/BPA Prophylaxis and Fitusiran Efficacy Periods

| | Inhibitor | | Non-Inhibitor | | Overall | |
|---|---|---|---|---|---|---|
| | Factor/BPA | Fitusiran 80 mg | Factor/BPA | Fitusiran 80 mg | Factor/BPA | Fitusiran 80 mg |
| | Prophylaxis | Prophylaxis | Prophylaxis | Prophylaxis | Prophylaxis | Prophylaxis |
| | (N = 19) | (N = 19) | (N = 46) | (N = 46) | (N = 65) | (N = 65) |
| Participants | 18 | 4 | 36 | 20 | 54 | 24 |
| with any treated | (94.7) | (21.1) | (78.3) | (43.5) | (83.1) | (36.9) |
| bleeding event, | | | | | | |
| n (%) | | | | | | |
| Estimated ABR | 11.405 | 2.318 | 5.888 | 3.155 | 7.482 | 2.908 |
| (95% CI) | (7.371, | (0.817, | (3.969, | (1.729, | (5.520, | (1.727, |
| | 17.647) | 6.573) | 8.733) | 5.758) | 10.141) | 4.898) |
| Rate ratio | | 0.203 | | 0.536 | | 0.389 |
| (95% CI)^{a} | | (0.074, | | (0.280, | | (0.224, |
| | | 0.562) | | 1.026) | | 0.675) |
| P-value^{b} | | 0.0021 | | 0.0598 | | 0.0008 |

^{a}Fitusiran rate divided by Factor/BPA Prophylaxis rate.
^{b}P-value from a repeated measures negative binomial regression model with study period (fitusiran efficacy period or factor/BPA prophylaxis period) as a fixed effect and a robust sandwich covariance matrix is constructed to account for the within subject dependence, the logarithm of the duration (in years) that each participant spends in each study period matching the bleeding episode data being analyzed as an offset variable (p-value versus null hypothesis of ratio = 1.)
The analysis is based on on-treatment strategy which included all treated bleeding events in the fitusiran efficacy period and the factor/BPA prophylaxis period and excluded any bleeding events in the period of intercurrent events.

TABLE 9
Observed Treated Bleeding Events and Duration of Follow-Up in
the Fitusiran Efficacy and the Factor/BPA Prophylaxis Periods

| Inhibitor | | Non-Inhibitor | | Overall | |
|---|---|---|---|---|---|
| Factor/BPA | Fitusiran 80 mg | Factor/BPA | Fitusiran 80 mg | Factor/BPA | Fitusiran 80 mg |
| Prophylaxis | Prophylaxis | Prophylaxis | Prophylaxis | Prophylaxis | Prophylaxis |
| (N = 19) | (N = 19) | (N = 46) | (N = 46) | (N = 65) | (N = 65) |

Duration of Follow-Up (Years)

| Number | 19 | | 19 | | 46 | | 46 | | 65 | | 65 | |
|---|---|---|---|---|---|---|---|---|---|---|---|---|
| Mean (SD) | 0.45 | (0.04) | 0.40 | (0.10) | 0.46 | (0.00) | 0.41 | (0.08) | 0.46 | (0.02) | 0.41 | (0.08) |

| | | | | | | |
|---|---|---|---|---|---|---|
| Median | 0.46 | 0.44 | 0.46 | 0.44 | 0.46 | 0.44 |
| Q1; Q3 | 0.46; 0.46 | 0.40; 0.44 | 0.46; 0.46 | 0.44; 0.44 | 0.46; 0.46 | 0.43; 0.44 |
| Min; Max | 0.29; 0.46 | 0.06; 0.44 | 0.44; 0.46 | 0.11; 0.44 | 0.29; 0.46 | 0.06; 0.44 |

Total Number of Treated Bleeding Events [n (%)]

| 0 | 1 | (5.3) | 15 | (78.9) | 10 | (21.7) | 26 | (56.5) | 11 | (16.9) | 41 | (63.1) |
|---|---|---|---|---|---|---|---|---|---|---|---|---|
| 1 | 5 | (26.3) | 1 | (5.3) | 12 | (26.1) | 10 | (21.7) | 17 | (26.2) | 11 | (16.9) |
| 2 | 3 | (15.8) | 0 | | 6 | (13.0) | 3 | (6.5) | 9 | (13.8) | 3 | (4.6) |
| 3 | 1 | (5.3) | 0 | | 5 | (10.9) | 3 | (6.5) | 6 | (9.2) | 3 | (4.6) |
| >3 | 9 | (47.4) | 3 | (15.8) | 13 | (28.3) | 4 | (8.7) | 22 | (33.8) | 7 | (10.8) |

Observed Annualized Bleeding Rate

| Number | 19 | | 19 | | 46 | | 46 | | 65 | | 65 | |
|---|---|---|---|---|---|---|---|---|---|---|---|---|
| Mean (SD) | 11.42 | (11.41) | 2.23 | (5.34) | 5.96 | (8.19) | 3.59 | (8.57) | 7.56 | (9.49) | 3.19 | (7.75) |

| | | | | | | |
|---|---|---|---|---|---|---|
| Median | 6.52 | 0.00 | 4.35 | 0.00 | 4.35 | 0.00 |
| Q1; Q3 | 2.17; 19.57 | 0.00; 0.00 | 2.17; 8.70 | 0.00; 2.65 | 2.17; 10.87 | 0.00; 2.25 |
| Min; Max | 0.00; 43.48 | 0.00; 19.88 | 0.00; 50.00 | 0.00; 51.26 | 0.00; 50.00 | 0.00; 51.26 |

The analysis is based on on-treatment strategy which included all treated bleeding events in the fitusiran efficacy period and the factor/BPA prophylaxis period, and excluded any bleeding events in the period of intercurrent events.

Key Secondary Endpoints

Annualized spontaneous bleeding rate in the fitusiran efficacy period and the factor or BPA prophylaxis period: Overall, for participants treated with 80 mg QM fitusiran prophylaxis the estimated spontaneous ABR was 2.222 (95% confidence interval [CI], 1.190 to 4.152) during the fitusiran efficacy period, and 5.002 (95% CI, 3.424 to 7.305) during the factor or BPA prophylaxis period, representing a statistically significant reduction of 55.6% (95% CI, 15.8% to 76.6%) in treated spontaneous bleeds in favor of fitusiran prophylaxis (P=0.0129). The estimated AsBR ratio was 0.444 (95% CI, 0.234 to 0.842). The mean and median of observed treated spontaneous bleeds are presented in Table 10 below.

TABLE 10
Mean and Median of Observed Treated Spontaneous Bleeds

| Observed Spontaneous | Factor/BPA | Fitusiran 80 mg |
|---|---|---|
| Annualized Bleeding | Prophylaxis | Prophylaxis |
| Rate | (N = 65) | (N = 65) |
| Mean (SD) | 5.09 (7.93) | 2.51 (7.33) |
| Median | 2.17 | 0.00 |
| Q1; Q3 | 0.00; 6.52 | 0.00; 2.25 |

Annualized joint bleeding rate in the fitusiran efficacy period and the factor or BPA prophylaxis period: Overall, for participants treated with 80 mg QM fitusiran prophylaxis the estimated joint ABR was 2.564 (95% confidence interval [CI], 1.440 to 4.566) during the fitusiran efficacy period, and 5.282 (95% C2, 3.647 to 7.651) during the factor or BPA prophylaxis period, representing a statistically significant reduction of 51.5% (95% CM, 9.0% to 74.1%) in treated joint bleeds in favor of fitusiran prophylaxis (P0.0242). The estimated AjBR ratio was 0.485 (95% CI, 0.259 to 0.910). The mean and median of observed treated joint bleeds are presented in Table 11.

TABLE 11
Mean and Median of Observed Treated Joint Bleeds

| | Factor/BPA | Fitusiran 80 mg |
|---|---|---|
| Observed Annualized | Prophylaxis | Prophylaxis |
| Bleeding Rate | (N = 65) | (N = 65) |
| Mean (SD) | 5.35 (8.19) | 2.82 (7.54) |
| Median | 2.17 | 0.00 |
| Q1; Q3 | 0.00; 6.52 | 0.00; 2.25 |

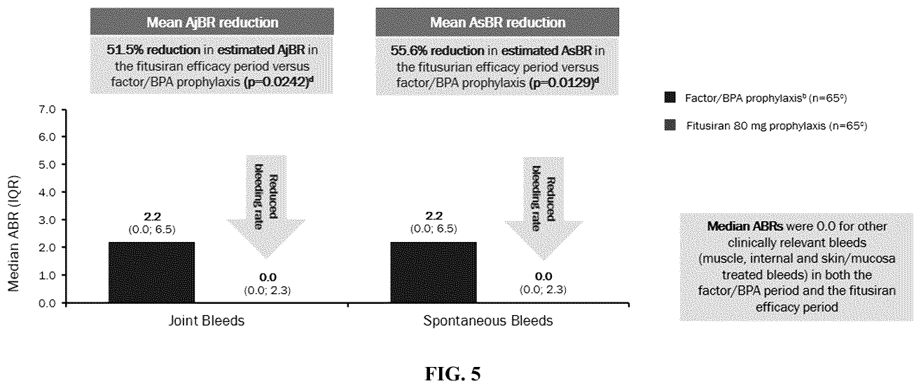

The data are also shown in Table 12 below and FIG. 5. The data are based on an on-treatment strategy, which included all treated bleeding events in the fitusiran efficacy period and the CFC/BPA prophylaxis period and excluded any bleeding events in the period of intercurrent events.

TABLE 12
Bleeding Events (Fitusiran Efficacy and Factor/BPA Prophylaxis Period*)

| | Factor/BPA | | Fitusiran 80 mg | | |
|---|---|---|---|---|---|
| | Prophylaxis | | Prophylaxis | | |
| | (N = 65^{†}) | | (N = 65^{†}) | | P-value^{‡} |
| Any treated bleeding event | | | | | |
| Estimated ABR (95% CI) | 7.5 | (5.5, 10.1) | 2.9 | (1.7, 4.9) | |
| % ABR reduction (95% CI) | | | 61.1 | (32.5, 77.6) | 0.0008 |
| Observed ABR Median | 4.4 | (2.2; 10.9) | 0.0 | (0.0; 2.3) | |
| (IQR) | | | | | |
| Observed ABR Mean (SD) | 7.6 | (9.5) | 3.2 | (7.8) | |
| Participants with zero | 11 | (16.9) | 41 | (63.1) | |
| treated bleeds, n (%) | | | | | |
| Treated spontaneous bleeds | | | | | |
| Estimated AsBR (95% CI) | 5.0 | (3.4, 7.3) | 2.2 | (1.2, 4.2) | |
| % AsBR reduction (95% CI) | | | 55.6 | (15.8, 76.6) | 0.0129 |
| Observed AsBR Median | 2.2 | (0.0; 6.5) | 0.0 | (0.0; 2.3) | |
| (IQR) | | | | | |
| Observed AsBR Mean (SD) | 5.1 | (7.9) | 2.5 | (7.3) | |
| Participants with zero | 23 | (35.4) | 46 | (70.8) | |
| spontaneous treated bleeds, | | | | | |
| n (%) | | | | | |
| Treated joint bleeds | | | | | |
| Estimated AjBR (95% CI) | 5.3 | (3.6, 7.7) | 2.6 | (1.4, 4.6) | |
| % AjBR reduction (95% CI) | | | 51.5 | (9.0, 74.1) | 0.0242 |
| Observed AjBR Median | 2.2 | (0.0; 6.5) | 0.0 | (0.0; 2.3) | |
| (IQR) | | | | | |
| Observed AjBR Mean (SD) | 5.4 | (8.2) | 2.8 | (7.5) | |
| Participants with zero joint | 22 | (33.8) | 44 | (67.7) | |
| treated bleeds, n (%) | | | | | |

*Fitusiran efficacy period (fitusiran prophylaxis) was defined as starting on Day 29 after the first dose of fitusiran up to Day 190, or the last day of bleeding follow up, whichever is the earliest. The factor/BPA prophylaxis period was defined as starting on Day −168 to Day −1, or the last day of bleeding follow up, whichever is the earliest. The factor/BPA prophylaxis period is defined as starting on day −168 to day −1, or the last day of bleeding follow up, whichever is the earliest.
^{†}Includes all participants who received factor/BPA prophylaxis and at least one dose of fitusiran before dose resumption (after the Sponsor initiated pause in dosing).
^{‡}P-value from a negative binomial regression model with study period (fitusiran efficacy period or factor/BPA prophylaxis period) as a fixed effect and a robust sandwich covariance matrix constructed to account for the within subject dependence, the logarithm of the duration (in years) that each participant spends in each study period matching the bleeding episode data being analyzed as an offset variable (p-value versus null hypothesis of ratio = 1.).

Annualized bleeding rate in the fitusiran onset period: In the 65 patients analyzed, the estimated annualized bleeding rate in the fitusiran onset period was 5.419 (95% CI, 3.716 to 7.901). The observed annualized bleeding rate in the fitusiran onset period was 5.42 (SD 8.28).

Annualized bleeding rate in the fitusiran treatment period: In the 65 patients analyzed, the estimated annualized bleeding rate in the fitusiran treatment period was 3.317 (95% CI, 2.111 to 5.211). The observed annualized bleeding rate in the fitusiran onset period was 3.48 (SD 6.98).

Consumption of factor concentrates and bypassing agents for management of breakthrough bleeds, including annualized weight-adjusted consumption of BPA and Factor: Annualized weight-adjusted BPA or Factor consumption was calculated for each participant during prophylaxis period as: [Sum of BPA dose per body weight received during corresponding period/number of days in corresponding period]*365.25. In this outcome measure, data of annualized weight-adjusted consumption of BPA or Factor were reported. The time frame was Factor/BPA prophylaxis period: Day −168 to Day −1 or up to last day of bleeding follow up (any day up to Day −1); 6-month fitusiran efficacy period: Day 29 to Day 190 or up to last day of bleeding follow up (any day up to Day 190), whichever was earliest. Consumption of BPA is summarized in Table 13. Consumption of Factor is summarized in Table 14.

TABLE 13
Annualized Weight-Adjusted Consumption of
BPA (Cohort A; Patients with Inhibitors)

| | Activated Prothrombin | | Recombinant Factor VIIa | |
|---|---|---|---|---|
| | Complex Concentrates | | (mcg/kg per participant | |
| | (U/kg per participant per year) | | per year) | |
| | BPA | Fituisran 80 mg | BPA | Fituisran 80 mg |
| | Prophylaxis | Prophylaxis | Prophylaxis | Prophylaxis |
| Number of | 15 | 15 | 7 | 7 |
| Participants | | | | |
| Analyzed | | | | |
| Annualized | 7912.7 | 39.7 | 18895.8 | 168.8 |
| Weight-adjusted | (5507.6) | (87.0) | (14081.9) | (290.8) |
| Consumption of | | | | |
| BPA; mean (SD) | | | | |

TABLE 14
Annualized Weight-Adjusted Consumption of Factor
(Cohort B; Patients Without Inhibitors)

| | | Factor | | Fitusiran 80 mg | |
|---|---|---|---|---|---|
| | | Prophylaxis | | Prophylaxis | |
| Overall Number of Participants | | 46 | | 46 | |
| Analyzed | | | | | |
| FVIII | Number of | 36 | | 36 | |
| | Participants Analyzed | | | | |
| | Mean (SD) IU/kg per | 3396.9 | (1144.5) | 60.7 | (148.3) |
| | participant per year | | | | |
| FIX | Number of | 10 | | 10 | |
| | Participants Analyzed | | | | |
| | Mean (SD) IU/kg per | 3175.5 | (961.3) | 17.8 | (56.1) |
| | participant per year | | | | |

Fewer participants required clotting factor concentrate (CFC) or BPA for breakthrough bleed treatment after switching to fitusiran. A total of 36 participants received CFC and 20 participants received BPA for breakthrough bleed treatment while on CFC/BPA prophylaxis. After switching to fitusiran, this reduced to 20 and 4 participants, respectively.

The total number of treated bleeds was lower in participants receiving fitusiran (total with inhibitors=18, total without inhibitors=54) compared to in participants receiving CFC (total=126) or BPA (total=101) prophylaxis.

Mean total weight-adjusted dose per bleed of CFC (FVIII=13.4 IU/kg, SD=5.5; FIX=26.2 IU/kg, SD=0.0) and BPA (aPCC=34.1 U/kg, SD=16.1; rFVIIa=38.2 μg/kg, SD=17.0) were markedly reduced when participants received fitusiran compared to CFC (FVIII=45.3 IU/kg, SD=41.8; FIX=73.6 IU/kg, SD=54.7) or BPA (aPCC=199.8 U/kg, SD=366.1; rFVIIa=709.9 μg/kg, SD=1163.8) prophylaxis.

Total mean consumption of FVIII and FIX for breakthrough bleeds was lower in patients receiving fitusiran prophylaxis compared to patients receiving CFC prophylaxis (Table 15). The total mean consumption of aPCC and rFVIIa for breakthrough bleeds was lower in participants receiving fitusiran prophylaxis compared to patients receiving BPA prophylaxis. In particular, annualized weight-adjusted consumption of aPCC for bleed therapy was 98.9% lower during fitusiran prophylaxis, annualized weight-adjusted consumption of rFVIIa was 96.8% lower during fitusiran prophylaxis, annualized weight-adjusted consumption of FVIII was 79.4% lower during fitusiran prophylaxis, and annualized weight-adjusted consumption of FIX was 93.8% lower during fitusiran prophylaxis.

Participants receiving fitusiran also required fewer injections to treat breakthrough bleeds (total with inhibitors=26, total without inhibitors=59) compared to those who received CFC (total=189) or BPA (total=419) prophylaxis. The number of treated bleeds in patients with inhibitors was 82.2% lower with fitusiran treatment compared to BPA prophylaxis, while the number of treated bleeds in patients without inhibitors was 57.1% during the fitusiran prophylaxis period compared with the factor/bypassing agent prophylaxis period. The total number of injections of BPA in patients with inhibitors was 93.8% lower with fitusiran treatment compared to BPA prophylaxis, and the total number of injections of replacement factor in patients without inhibitors was 68.8% lower during the fitusiran prophylaxis period compared with the factor/bypassing agent prophylaxis period.

TABLE 15
Consumption of CFC/BPA for Treatment of Breakthrough Bleeds in the
Fitusiran Efficacy Period vs CFC/BPA Prophylaxis Period (EAS 1*)

| | Inhibitor | | | | Non-inhibitor | | | |
|---|---|---|---|---|---|---|---|---|
| | | | Fitusiran 80 mg | | CFC | | Fitusiran 80 mg | |
| | BPA prophylaxis | | prophylaxis | | prophylaxis | | prophylaxis | |
| | (n = 19**) | | (n = 19**) | | (n = 46***) | | (n = 46***) | |
| | aPCC | rFVIIa | aPCC | rFVIIa | FVIII | FIX | FVIII | FIX |
| | (U/kg) | (μg/kg) | (U/kg) | (μg/kg) | (IU/kg) | (IU/kg) | (IU/kg) | (IU/kg) |
| Event | (n = 15) | (n = 5) | (n = 2) | (n = 2) | (n = 29) | (n = 7) | (n = 19) | (n = 1) |
| Annualized | 2353.0 | 7468.7 | 25.6 | 236.4 | 294.5 | 288.6 | 60.7 | 17.8 |
| mean weight- | (4317.0) | (5756.6) | (82.6) | (327.0) | (366.6) | (402.9) | (148.3) | (56.1) |
| adjusted | | | | | | | | |
| consumption | | | | | | | | |
| for bleed | | | | | | | | |
| therapy, unit | | | | | | | | |
| (SD) | | | | | | | | |
| Total number | 77 | 24 | 5 | 13 | 108 | 18 | 51 | 3 |
| of treated | | | | | | | | |
| bleeds, n | | | | | | | | |
| Total number | 18 (94.7) | | 4 (21.1) | | 36 (78.3) | | 20 (43.5) | |
| of participants | | | | | | | | |
| with any | | | | | | | | |
| treated bleed, † | | | | | | | | |
| n (%) | | | | | | | | |
| Mean total | 199.8 | 709.9 | 34.1 | 38.2 | 45.3 | 73.6 | 13.4 | 26.2 |
| weight-adjusted | (366.1) | (1163.8) | (16.1) | (17.0) | (41.8) | (54.7) | (5.5) | (0.0) |
| dose per bleed, | | | | | | | | |
| unit (SD) | | | | | | | | |
| Total number | 260 | 159 | 7 | 19 | 159 | 30 | 56 | 3 |
| of injections, n | | | | | | | | |

aPCC, prothrombin complex concentrate; BPA, bypassing agent; CFC, clotting factor concentrate; EAS 1, efficacy analysis set 1; rFVIIa, recombinant activated factor VII.
*Includes all participants who received CFC/BPA prophylaxis and at least one dose of 80 mg fitusiran before dose resumption (after the Sponsor initiated pause in dosing).
**Participants with inhibitors who received BPA prophylaxis and at least one dose of fitusiran before dose resumption; a total number of participants, which includes the number of participants with breakthrough bleeds.
***Participants without inhibitors who received factor prophylaxis and at least one dose of fitusiran before dose resumption; a total number of participants, which includes the number of participants with breakthrough bleeds.

Overall, the consumption endpoint results consistently favored fitusiran prophylaxis over CFC/BPA prophylaxis. Fitusiran prophylaxis reduced the total CFC/BPA consumption in patients by reducing the mean total weight-adjusted dose per bleed, the number of treated bleeds, and the number of injections required to treat bleeds, thereby reducing treatment burden in patients with hemophilia A or B with and without inhibitors.

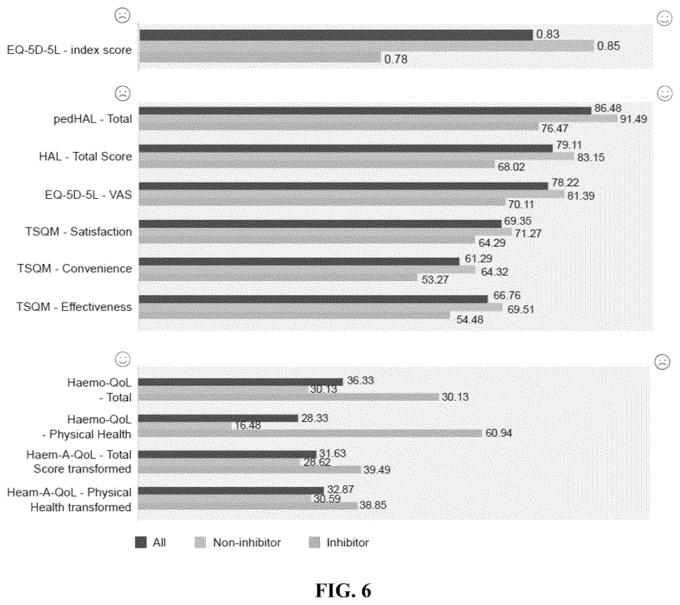

Change in Haem-A-QOL, EO-5D-5L. HAL, and TSOM-9 scores: At enrollment (month −6) people without inhibitors had generally less impaired PRO scores compared to people with inhibitors (FIG. 6).

For Ham-A-QoL, change from baseline (month −6) for both treatments was compared via a mixed model for repeated measurements (MMRM): change from month −6 to month 7 for fitusiran prophylaxis vs change from month −6 to day 1 for factor/BPA prophylaxis. Change from month −6 to day 1 and change from month −6 to month 7 were the response variables. Study period (factor/BPA prophylaxis period and fitusiran treatment period) and score at month −6 were fixed effects and a robust sandwich covariance matrix was constructed to account for the within-subject dependence.

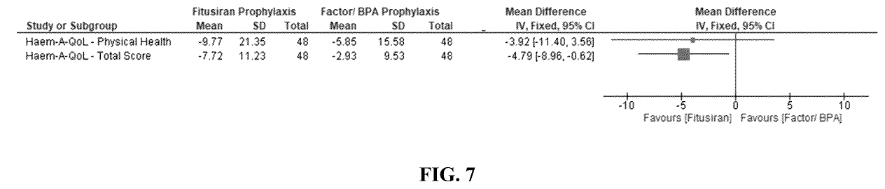

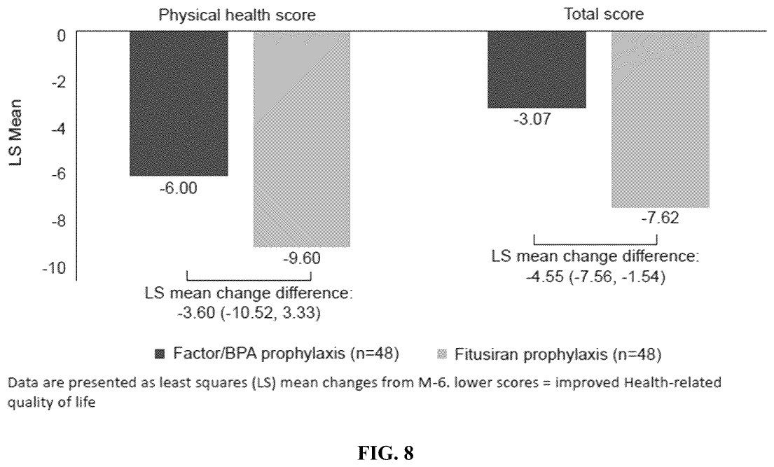

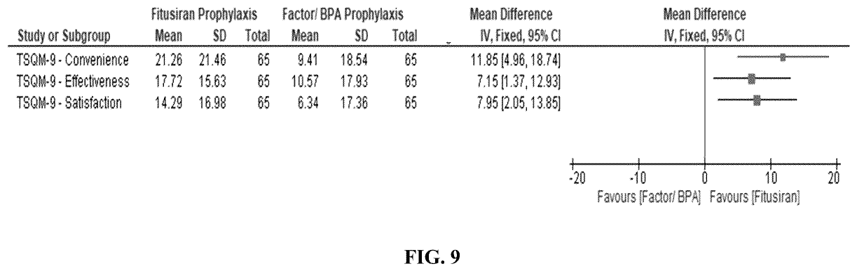

At enrolment (month −6), transformed Haem-A-QoL physical health and total scores were, respectively, 32.87 (SD 23.68) and 31.63 (SD 18.54). The Least Square (LS) mean (95% CI) of the change in transformed total score from baseline (i.e., difference between change from month −6 to baseline (day 1) and the change from month −6 to month 7) in Haem-A-QoL scores was −7.62 (−10.26 to −4.98) for fitusiran prophylaxis and −3.07 (−5.56 to −0.58) for factor/BPA prophylaxis. The LS mean difference was −4.55 (95% CI, −7.56 to −1.54) and significantly reduced in favor of fitusiran (P=0.0039) (FIGS. 7 and 8).

The LS mean (95% CI) of the change in transformed physical health domain score from baseline was −9.60 (−15.35, −3.84) for fitusiran prophylaxis and −6.00 (−10.19, −1.81) for factor/BPA prophylaxis (Table 16). The least squares (LS) mean difference between change from month −6 to baseline (day 1) and the change from month −6 to month 7 in Haem-A-QoL scores demonstrated a nominal improvement in physical health score in favor of fitusiran (−3.60 [95%-CI: −10.52, 3.33]) (FIGS. 7 and 8).

TABLE 16
Change in Haem-A-QoL Scores

| | | |
|---|---|---|
| | Factor/BPA | Fitusiran 80 mg |
| | Prophylaxis | Prophylaxis |
| Domain | (N = 48) | (N = 48) |

Transformed Total Score

| | | | | |
|---|---|---|---|---|
| LS Mean (95% CI) | −3.07 | (−5.56, −0.58) | −7.62 | (−10.26, −4.98) |
| LS Mean difference | | | −4.55 | (−7.56, −1.54) |
| (95% CI) | | | | |
| vs Factor/BPA | | | | |
| prophylaxis period | | | | |

| | |
|---|---|
| P-value | 0.0039 |

Transformed Physical Health Score

| | | | | |
|---|---|---|---|---|
| LS Mean (95% CI) | −6.00 | (−10.19, −1.81) | −9.60 | (−15.35, −3.84) |
| LS Mean difference | | | −3.60 | (−10.52, 3.33) |
| (95% CI) | | | | |
| vs Factor/BPA | | | | |
| prophylaxis period | | | | |

| | |
|---|---|
| P-value | 0.3008 |

Changes in TSQM-9, EQ-5D-5L, and HAL from month −6 (enrollment) to day 1 and month 7 were summarized descriptively.

At enrollment (month −6), the EQ-5D-5L index score was 0.83 (SD 0.13). Fitusiran also demonstrated nominal improvements in the EQ-5D-5L index score compared with factor/BPA prophylaxis, with a mean change from month −6 to month 7 of 0.04 (SD 0.13) compared to a mean change from month −6 to day 1 of 0.0 (SD 0.13).

At enrollment (month −6), the HAL total score was 79.11 (SD 20.29). In addition to showing a good functional ability to perform activities of daily living, patients rated an even better physical ability over the two periods of assessment of the study as evidenced by changes in HAL scores: mean changes in total score were 3.05 (SD 20.04) and 2.45 (SD 19.42) for the fitusiran and factor/BPA prophylaxis arms, respectively.

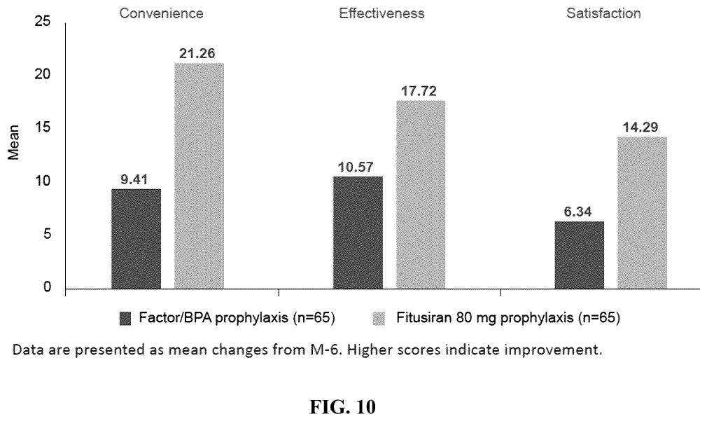

At enrollment (month −6), the TSQM-9 baseline scores for effectiveness, convenience and satisfaction were 66.76 (SD 18.06), 61.29 (SD 17.97) and 69.35 (SD 15.91), respectively. Overall results of the mean TSQM-9 score consistently favored fitusiran prophylaxis versus factor/BPA prophylaxis in all three domains of effectiveness, convenience, and global satisfaction (FIG. 7). Overall, mean TSQM-9 scores consistently favored fitusiran prophylaxis in comparison with factor/BPA prophylaxis in all three domains of effectiveness, convenience, and global satisfaction (FIG. 10).

Safety Results: Sixty-seven (83.8%) participants were enrolled, received at least 1 dose of fitusiran before dose resumption (after the Sponsor initiated pause in dosing), and were included in the Safety Analysis Set 1. Overall, 22 (33.8%) participants in the factor/BPA prophylaxis period and 48 (71.6%) participants in the fitusiran prophylaxis period experienced at least 1 adverse event (AE).

A total of five serious adverse events (SAEs) were reported in 5 (7.7%) participants in the factor/BPA prophylaxis period and 13 SAEs were reported in 9 (13.4%) participants in the fitusiran prophylaxis period. The most common SAE in the fitusiran prophylaxis period was haemophilic arthropathy (2 [3.0%] participants); all other SAEs in the fitusiran prophylaxis period were reported in 1 (1.5%) participant each.

Two (3.1%) participants in the factor/BPA prophylaxis period and 22 (32.8%) participants in the fitusiran prophylaxis period experienced adverse events of special interest (AESIs). In the fitusiran prophylaxis period, these included two (3.0%) participants with suspected or confirmed thromboembolic events (cerebrovascular accident and suspected thrombosis [thrombosis on papilla of left eye]). The participant with cerebrovascular accident had a history of a right lower extremity deep vein thrombosis not known by the Investigator at the time of enrollment (exclusion criterion).

17 (25.4%) participants with ALT or AST elevations >3×ULN. One of these participants experienced laboratory abnormalities consistent with Hy's Law, with resolution following the final dose of fitusiran. Five (7.5%) participants with cholecystitis and 5 (7.5%) participants with cholelithiasis, including 2 participants with both events. Two (3.0%) participants in the fitusiran prophylaxis period experienced AEs that resulted in study drug discontinuation (cerebrovascular accident and abdominal discomfort).

There were no fatal AEs reported. A summary of AEs is provided in Tables 17-21 below.

TABLE 17
Select Safety Results from Clinical Study

| | Factor/BPA | | Fitusiran 80 mg | |
|---|---|---|---|---|
| | Prophylaxis | | Prophylaxis | |
| Event, n (%) | (N = 65) | | (N = 67*) | |
| Participants with any AE | 22 | (33.8) | 48 | (71.6) |
| Participants with Any SAE | 5 | (7.7) | 9 | (13.4) |
| Participants with most common | | | | |
| SAEs^{†} | | | | |
| Haemophilic arthropathy | 2 | (3.1) | 2 | (3.0) |
| Participants with any AESI | 2 | (3.1) | 22 | (32.8) |
| Suspected or confirmed | 0 | | 2 | (3.0) |
| thromboembolic events^{‡} | | | | |
| ALT or AST elevations >3 × ULN^{§} | 2 | (3.1) | 17 | (25.4) |
| Cholecystitis | 0 | | 5 | (7.5) |
| Cholelithiasis | 0 | | 5 | (7.5) |
| Participants with AEs leading to | — | | 2 | (3.0) |
| fitusiran prophylaxis discontinuation | | | | |
| (cerebrovascular accident and | | | | |
| abdominal discomfort) | | | | |
| Participants with any AE leading to | 0 | | 0 | |
| death | | | | |

*Includes all participants who enrolled and then received at least one dose of fitusiran before dose resumption (after the Sponsor initiated pause in dosing);
^{†}All other SAEs were reported in 1 (1.5%) participant each;
^{‡}Includes AEs of cerebrovascular accident and thrombosis (verbatim: thrombosis on papilla of left eye). The participant with cerebrovascular accident had a history of right lower extremity deep vein thrombosis not known by the Investigator at the time of enrolment (exclusion criterion);
^{§}One of these participants experienced laboratory abnormalities consistent with Hy's Law, with resolution following the final dose of fitusiran.
AE, adverse event; AESI, adverse event of special interest; ALT, alanine aminotransferase; AST, aspartate transaminase; SAE, serious adverse event; ULN, upper limit of normal.

TABLE 18
Serions Adverse Events in Patients Treated with Fitusiran 80 mg QM

| | Cohort A: | | Cohort A: | | Cohort B: | |
|---|---|---|---|---|---|---|
| | SAS I - BPA | | SAS I - Fitusiran | | SAS I - Factor | |
| | Prophylaxis | | 80 mg QM | | Prophylaxis | |
| | Affected/At | # | Affected/At | # | Affected/At | # |
| | Risk (%) | Events | Risk (%) | Events | Risk (%) | Events |
| Total | 5/19 | | 5/21 | | 0/46 | |
| | (26.32%) | | (23.81%) | | (0%) | |

Gastrointestinal disorders

| | | | | | | |
|---|---|---|---|---|---|---|
| Pancreatitis Acute | 0/19 | 0 | 1/21 | 1 | 0/46 | 0 |
| | (0%) | | (4.76%) | | (0%) | |

Hepatobiliary disorders

| | | | | | | |
|---|---|---|---|---|---|---|
| Cholelithiasis | 0/19 | 0 | 1/21 | 1 | 0/46 | 0 |
| | (0%) | | (4.76%) | | (0%) | |

Infections and infestations

| | | | | | | |
|---|---|---|---|---|---|---|
| Covid-19 | 0/19 | 0 | 0/21 | 0 | 0/46 | 0 |
| Pneumonia | (0%) | | (0%) | | (0%) | |
| Gastroenteritis | 1/19 | 1 | 0/21 | 0 | 0/46 | 0 |
| | (5.26%) | | (0%) | | (0%) | |
| Vascular Device | 0/19 | 0 | 0/21 | 0 | 0/46 | 0 |
| Infection | (0%) | | (0%) | | (0%) | |

Injury, poisoning and procedural complications

| | | | | | | |
|---|---|---|---|---|---|---|
| Fall | 0/19 | 0 | 1/21 | 1 | 0/46 | 0 |
| | (0%) | | (4.76%) | | (0%) | |
| Femur Fracture | 0/19 | 0 | 1/21 | 1 | 0/46 | 0 |
| | (0%) | | (4.76%) | | (0%) | |

Investigations

| | | | | | | |
|---|---|---|---|---|---|---|
| C-Reactive Protein | 0/19 | 0 | 0/21 | 0 | 0/46 | 0 |
| Increased | (0%) | | (0%) | | (0%) | |

Musculoskeletal and connective tissue disorders

| | | | | | | |
|---|---|---|---|---|---|---|
| Haemarthrosis | 1/19 | 1 | 0/21 | 0 | 0/46 | 0 |
| | (5.26%) | | (0%) | | (0%) | |
| Haemophilic | 2/19 | 2 | 1/21 | 1 | 0/46 | 0 |
| Arthropathy | (10.53%) | | (4.76%) | | (0%) | |
| Muscle | 1/19 | 1 | 0/21 | 0 | 0/46 | 0 |
| Haemorrhage | (5.26%) | | (0%) | | (0%) | |

Neoplasms benign, malignant and unspecified (incl cysts and polyps)

| | | | | | | |
|---|---|---|---|---|---|---|
| Biliary Neoplasm | 0/19 | 0 | 0/21 | 0 | 0/46 | 0 |
| | (0%) | | (0%) | | (0%) | |

Nervous system disorders

| | | | | | | |
|---|---|---|---|---|---|---|
| Cerebrovascular | 0/19 | 0 | 0/21 | 0 | 0/46 | 0 |
| Accident | (0%) | | (0%) | | (0%) | |

Respiratory, thoracic and mediastinal disorders

| | | | | | | |
|---|---|---|---|---|---|---|
| Asthma Late Onset | 0/19 | 0 | 0/21 | 0 | 0/46 | 0 |
| | (0%) | | (0%) | | (0%) | |

Skin and subcutaneous tissue disorders

| | | | | | | |
|---|---|---|---|---|---|---|
| Stevens-Johnson | 0/19 | 0 | 0/21 | 0 | 0/46 | 0 |
| Syndrome | (0%) | | (0%) | | (0%) | |

Surgical and medical procedures

| Central Venous | 0/19 | 0 | 1/21 | 1 | 0/46 | 0 |
|---|---|---|---|---|---|---|
| Catheter | (0%) | | (4.76%) | | (0%) | |
| Removal | | | | | | |
| | Cohort B: | | Overall: | | Overall: | |
| | SAS I - Fitusiran | | SAS I - Factor/BPA | | SAS I - Fitusiran | |
| | 80 mg QM | | Prophylaxis | | 80 mg QM | |
| | Affected/At | # | Affected/At | # | Affected/At | # |
| | Risk (%) | Events | Risk (%) | Events | Risk (%) | Events |
| Total | 4/46 | | 5/65 | | 9/67 | |
| | (8.7%) | | (7.69%) | | (13.43%) | |

Gastrointestinal disorders

| | | | | | | |
|---|---|---|---|---|---|---|
| Pancreatitis Acute | 0/46 | 0 | 0/65 | 0 | 1/67 | 1 |
| | (0%) | | (0%) | | (1.49%) | |

Hepatobiliary disorders

| | | | | | | |
|---|---|---|---|---|---|---|
| Cholelithiasis | 0/46 | 0 | 0/65 | 0 | 1/67 | 1 |
| | (0%) | | (0%) | | (1.49%) | |

Infections and infestations

| | | | | | | |
|---|---|---|---|---|---|---|
| Covid-19 | 0/46 | 0 | 0/65 | 0 | 0/67 | 0 |
| Pneumonia | (0%) | | (0%) | | (0%) | |
| Gastroenteritis | 0/46 | 0 | 1/65 | 1 | 0/67 | 0 |
| | (0%) | | (1.54%) | | (0%) | |
| Vascular Device | 1/46 | 1 | 0/65 | 0 | 1/67 | 1 |
| Infection | (2.17%) | | (0%) | | (1.49%) | |

Injury, poisoning and procedural complications

| | | | | | | |
|---|---|---|---|---|---|---|
| Fall | 0/46 | 0 | 0/65 | 0 | 1/67 | 1 |
| | (0%) | | (0%) | | (1.49%) | |
| Femur Fracture | 0/46 | 0 | 0/65 | 0 | 1/67 | 1 |
| | (0%) | | (0%) | | (1.49%) | |

Investigations

| | | | | | | |
|---|---|---|---|---|---|---|
| C-Reactive Protein | 1/46 | 1 | 0/65 | 0 | 1/67 | 1 |
| Increased | (2.17%) | | (0%) | | (1.49%) | |

Musculoskeletal and connective tissue disorders

| | | | | | | |
|---|---|---|---|---|---|---|
| Haemarthrosis | 0/46 | 0 | 1/65 | 1 | 0/67 | 0 |
| | (0%) | | (1.54%) | | (0%) | |
| Haemophilic | 1/46 | 1 | 2/65 | 2 | 2/67 | 2 |
| Arthropathy | (2.17%) | | (3.08%) | | (2.99%) | |
| Muscle | 0/46 | 0 | 1/65 | 1 | 0/67 | 0 |
| Haemorrhage | (0%) | | (1.54%) | | (0%) | |

Neoplasms benign, malignant and unspecified (incl cysts and polyps)

| | | | | | | |
|---|---|---|---|---|---|---|
| Biliary Neoplasm | 1/46 | 1 | 0/65 | 0 | 1/67 | 1 |
| | (2.17%) | | (0%) | | (1.49%) | |

Nervous system disorders

| | | | | | | |
|---|---|---|---|---|---|---|
| Cerebrovascular | 1/46 | 1 | 0/65 | 0 | 1/67 | 1 |
| Accident | (2.17%) | | (0%) | | (1.49%) | |

Respiratory, thoracic and mediastinal disorders

| | | | | | | |
|---|---|---|---|---|---|---|
| Asthma Late Onset | 1/46 | 1 | 0/65 | 0 | 1/67 | 1 |
| | (2.17%) | | (0%) | | (1.49%) | |

Skin and subcutaneous tissue disorders

| | | | | | | |
|---|---|---|---|---|---|---|
| Stevens-Johnson | 1/46 | 1 | 0/65 | 0 | 1/67 | 1 |
| Syndrome | (2.17%) | | (0%) | | (1.49%) | |

Surgical and medical procedures

| | | | | | | |
|---|---|---|---|---|---|---|
| Central Venous | 0/46 | 0 | 0/65 | 0 | 1/67 | 1 |
| Catheter | (0%) | | (0%) | | (1.49%) | |
| Removal | | | | | | |

TABLE 19
Serious Adverse Events in Patients Treated with Fitusiran 50 mg Q2M

| | Cohort A: SAS 2 - | | Cohort A: SAS 2 - | |
|---|---|---|---|---|
| | BPA Prophylaxis | | Fitusiran 50 mg Q2M | |
| | Affected/At | | Affected/At | |
| | Risk (%) | # Events | Risk (%) | # Events |
| Total | 1/2 (50%) | | 1/2 (50%) | |

Gastrointestinal disorders

| | | | | |
|---|---|---|---|---|
| Pancreatitis Acute | 0/2 (0%) | 0 | 0/2 (0%) | 0 |

Hepatobiliary disorders

| | | | | |
|---|---|---|---|---|
| Cholelithiasis | 0/2 (0%) | 0 | 0/2 (0%) | 0 |

Infections and infestations

| | | | | |
|---|---|---|---|---|
| Covid-19 Pneumonia | 0/2 (0%) | 0 | 1/2 (50%) | 1 |
| Gastroenteritis | 0/2 (0%) | 0 | 0/2 (0%) | 0 |
| Vascular Device Infection | 0/2 (0%) | 0 | 0/2 (0%) | 0 |

Injury, poisoning and procedural complications

| | | | | |
|---|---|---|---|---|
| Fall | 0/2 (0%) | 0 | 0/2 (0%) | 0 |
| Femur Fracture | 0/2 (0%) | 0 | 0/2 (0%) | 0 |

Investigations

| | | | | |
|---|---|---|---|---|
| C-Reactive Protein Increased | 0/2 (0%) | 0 | 0/2 (0%) | 0 |

Musculoskeletal and connective tissue disorders

| | | | | |
|---|---|---|---|---|
| Haemarthrosis | 1/2 (50%) | 0 | 0/2 (0%) | 0 |
| Haemophilic Arthropathy | 0/2 (0%) | 0 | 0/2 (0%) | 0 |
| Muscle Haemorrhage | 0/2 (0%) | 0 | 0/2 (0%) | 0 |

Neoplasmis benign, malignant and unspecified (incl cysts and polyps)

| | | | | |
|---|---|---|---|---|
| Biliary Neoplasm | 0/2 (0%) | 0 | 0/2 (0%) | 0 |

Nervous system disorders

| | | | | |
|---|---|---|---|---|
| Cerebrovascular Accident | 0/2 (0%) | 0 | 0/2 (0%) | 0 |

Respiratory, thoracic and mediastinal disorders

| | | | | |
|---|---|---|---|---|
| Asthma Late Onset | 0/2 (0%) | 0 | 0/2 (0%) | 0 |

Skin and subcutaneous tissue disorders

| | | | | |
|---|---|---|---|---|
| Stevens-Johnson Syndrome | 0/2 (0%) | 0 | 0/2 (0%) | 0 |

Surgical and medical procedures

| | | | | |
|---|---|---|---|---|
| Central Venous Catheter Removal | 0/2 (0%) | 0 | 0/2 (0%) | 0 |

TABLE 20
Other Adverse Events in Patients Treated with Fitusiran 80 mg QM

| | Cohort A: | | Cohort A: | | Cohort B: | |
|---|---|---|---|---|---|---|
| | SAS I - BPA | | SAS I - Fitusiran | | SAS I - Factor | |
| | Prophylaxis | | 80 mg QM | | Prophylaxis | |
| | Affected/At | # | Affected/At | # | Affected/ | # |
| | Risk (%) | Events | Risk (%) | Events | At Risk (%) | Events |
| Total | 10/19 | | 13/21 | | 9/46 | |
| | (52.63%) | | (61.9%) | | (19.57%) | |

Blood and lymphatic system disorders

| | | | | | | |
|---|---|---|---|---|---|---|
| Anaemia | 2/19 | 2 | 0/21 | 0 | 0/46 | 0 |
| | (10.53%) | | (0%) | | (0%) | |

Gastrointestinal disorders

| | | | | | | |
|---|---|---|---|---|---|---|
| Abdominal Pain | 0/19 | 0 | 2/21 | 3 | 0/46 | 0 |
| | (0%) | | (9.52%) | | (0%) | |
| Dental Caries | 1/19 | 2 | 0/21 | 0 | 1/46 | 1 |
| | (5.26%) | | (0%) | | (2.17%) | |
| Eosinophilic | 1/19 | 1 | 0/21 | 0 | 0/46 | 0 |
| Oesophagitis | (5.26%) | | (0%) | | (0%) | |
| Gastrointestinal Motility | 1/19 | 1 | 0/21 | 0 | 0/46 | 0 |
| Disorder | (5.26%) | | (0%) | | (0%) | |
| Haemorrhoids | 0/19 | 0 | 0/21 | 0 | 0/46 | 0 |
| | (0%) | | (0%) | | (0%) | |
| Nausea | 1/19 | 1 | 0/21 | 0 | 0/46 | 0 |
| | (5.26%) | | (0%) | | (0%) | |
| Teething | 1/19 | 1 | 0/21 | 0 | 0/46 | 0 |
| | (5.26%) | | (0%) | | (0%) | |
| Toothache | 0/19 | 0 | 1/21 | 1 | 0/46 | 0 |
| | (0%) | | (4.76%) | | (0%) | |
| Vomiting | 1/19 | 1 | 1/21 | 1 | 0/46 | 0 |
| | (5.26%) | | (4.76%) | | (0%) | |

General disorders

| | | | | | | |
|---|---|---|---|---|---|---|
| Injection Site Erythema | 0/19 | 0 | 2/21 | 2 | 0/46 | 0 |
| | (0%) | | (9.52%) | | (0%) | |
| Injection Site Pain | 0/19 | 0 | 2/21 | 2 | 0/46 | 0 |
| | (0%) | | (9.52%) | | (0%) | |

Hepatobiliary disorders

| | | | | | | |
|---|---|---|---|---|---|---|
| Cholecystitis | 0/19 | 0 | 2/21 | 2 | 0/46 | 0 |
| | (0%) | | (9.52%) | | (0%) | |
| Cholelithiasis | 0/19 | 0 | 2/21 | 2 | 0/46 | 0 |
| | (0%) | | (9.52%) | | (0%) | |
| Hepatic Steatosis | 0/19 | 0 | 2/21 | 2 | 1/46 | 1 |
| | (0%) | | (9.52%) | | (2.17%) | |

Infections and infestations

| | | | | | | |
|---|---|---|---|---|---|---|
| Genital Herpes Simplex | 1/19 | 1 | 0/21 | 0 | 0/46 | 0 |
| | (5.26%) | | (0%) | | (0%) | |
| Influenza | 1/19 | 1 | 1/21 | 1 | 2/46 | 2 |
| | (5.26%) | | (4.76%) | | (4.35%) | |
| Nasopharyngitis | 0/19 | 0 | 1/21 | 1 | 1/46 | 1 |
| | (0%) | | (4.76%) | | (2.17%) | |
| Tinea Pedis | 1/19 | 1 | 0/21 | 0 | 1/46 | 1 |
| | (5.26%) | | (0%) | | (2.17%) | |
| Upper Respiratory Tract | 3/19 | 4 | 4/21 | 5 | 1/46 | 1 |
| Infection | (15.79%) | | (19.05%) | | (2.17%) | |

Injury, poisoning and procedural complications

| | | | | | | |
|---|---|---|---|---|---|---|
| Arthropod Bite | 1/19 | 1 | 1/21 | 1 | 0/46 | 0 |
| | (5.26%) | | (4.76%) | | (0%) | |
| Buttock Injury | 1/19 | 1 | 0/21 | 0 | 0/46 | 0 |
| | (5.26%) | | (0%) | | (0%) | |
| Fall | 2/19 | 2 | 0/21 | 0 | 0/46 | 0 |
| | (10.53%) | | (0%) | | (0%) | |

Investigations

| | | | | | | |
|---|---|---|---|---|---|---|
| Alanine | 1/19 | 3 | 3/21 | 3 | 0/46 | 0 |
| Aminotransferase | (5.26%) | | (14.29%) | | (0%) | |
| Increased | | | | | | |
| Aspartate | 1/19 | 1 | 1/21 | 3 | 0/46 | 0 |
| Aminotransferase | (5.26%) | | (4.76%) | | (0%) | |
| Increased | | | | | | |
| Fibrin D Dimer | 0/19 | 0 | 3/21 | 3 | 0/46 | 0 |
| Increased | (0%) | | (14.29%) | | (0%) | |
| Lymphocyte Count | 0/19 | 0 | 0/21 | 0 | 0/46 | 0 |
| Increased | (0%) | | (0%) | | (0%) | |
| Neutrophil Count | 0/19 | 0 | 0/21 | 0 | 0/46 | 0 |
| Decreased | (0%) | | (0%) | | (0%) | |
| White Blood Cell Count | 0/19 | 0 | 0/21 | 0 | 0/46 | 0 |
| Decreased | (0%) | | (0%) | | (0%) | |

Metabolism and nutrition disorders

| | | | | | | |
|---|---|---|---|---|---|---|
| Iron Deficiency | 1/19 | 1 | 0/21 | 0 | 0/46 | 0 |
| | (5.26%) | | (0%) | | (0%) | |

Musculoskeletal and connective tissue disorders

| | | | | | | |
|---|---|---|---|---|---|---|
| Arthralgia | 1/19 | 1 | 1/21 | 1 | 3/46 | 4 |
| | (5.26%) | | (4.76%) | | (6.52%) | |
| Joint Swelling | 1/19 | 1 | 0/21 | 0 | 0/46 | 0 |
| | (5.26%) | | (0%) | | (0%) | |
| Synovitis | 1/19 | 1 | 1/21 | 1 | 0/46 | 0 |
| | (5.26%) | | (4.76%) | | (0%) | |

Nervous system disorders

| | | | | | | |
|---|---|---|---|---|---|---|
| Headache | 2/19 | 2 | 1/21 | 2 | 1/46 | 1 |
| | (10.53%) | | (4.76%) | | (2.17%) | |

Respiratory, thoracic and mediastinal disorders

| | | | | | | |
|---|---|---|---|---|---|---|
| Cough | 0/19 | 0 | 2/21 | 3 | 0/46 | 0 |
| | (0%) | | (9.52%) | | (0%) | |

Skin and subcutaneous tissue disorders

| Dermatitis Allergic | 1/19 | 1 | 0/21 | 0 | 0/46 | 0 |
|---|---|---|---|---|---|---|
| | (5.26%) | | (0%) | | (0%) | |
| Pruritus | 1/19 | 1 | 0/21 | 0 | 0/46 | 0 |
| | (5.26%) | | (0%) | | (0%) | |
| | Cohort B: | | Overall: | | Overall: | |
| | SAS I Fitusiran | | SAS I - Factor BPA | | SAS I - Fitusiran | |
| | 80 mg QM | | Prophylaxis | | 80 mg QM | |
| | Affected/At | # | Affected/At | # | Affected/At | # |
| | Risk (%) | Events | Risk (%) | Events | Risk (%) | Events |
| Total | 28/46 | | 19/65 | | 41/67 | |
| | (60.87%) | | (29.23%) | | (61.19%) | |

Blood and lymphatic system disorders

| | | | | | | |
|---|---|---|---|---|---|---|
| Anaemia | 0/46 | 0 | 2/65 | 2 | 0/67 | 0 |
| | (0%) | | (3.08%) | | (0%) | |

Gastrointestinal disorders

| | | | | | | |
|---|---|---|---|---|---|---|
| Abdominal Pain | 1/46 | 1 | 0/65 | 0 | 3/67 | 4 |
| | (2.17%) | | (0%) | | (4.48%) | |
| Dental Caries | 0/46 | 0 | 2/65 | 3 | 0/67 | 0 |
| | (0%) | | (3.08%) | | (0%) | |
| Eosinophilic | 0/46 | 0 | 1/65 | 1 | 0/67 | 0 |
| Oesophagitis | (0%) | | (1.54%) | | (0%) | |
| Gastrointestinal Motility | 0/46 | 0 | 1/65 | 1 | 0/67 | 0 |
| Disorder | (0%) | | (1.54%) | | (0%) | |
| Haemorrhoids | 0/46 | 0 | 0/65 | 0 | 0/67 | 0 |
| | (0%) | | (0%) | | (0%) | |
| Nausea | 0/46 | 0 | 1/65 | 1 | 0/67 | 0 |
| | (0%) | | (1.54%) | | (0%) | |
| Teething | 0/46 | 0 | 1/65 | | 0/67 | 0 |
| | (0%) | | (1.54%) | | (0%) | |
| Toothache | 1/46 | 1 | 0/65 | 0 | 2/67 | 2 |
| | (2.17%) | | (0%) | | (2.99%) | |
| Vomiting | 0/46 | 0 | 1/65 | 1 | 1/67 | 1 |
| | (0%) | | (1.54%) | | (1.49%) | |

General disorders

| | | | | | | |
|---|---|---|---|---|---|---|
| Injection Site Erythema | 1/46 | 2 | 0/65 | 0 | 3/67 | 4 |
| | (2.17%) | | (0%) | | (4.48%) | |
| Injection Site Pain | 3/46 | 3 | 0/65 | 0 | 5/67 | 5 |
| | (6.52%) | | (0%) | | (7.46%) | |

Hepatobiliary disorders

| | | | | | | |
|---|---|---|---|---|---|---|
| Cholecystitis | 2/46 | 2 | 0/65 | 0 | 4/67 | 4 |
| | (4.35%) | | (0%) | | (5.97%) | |
| Cholelithiasis | 3/46 | 3 | 0/65 | 0 | 5/67 | 5 |
| | (6.52%) | | (0%) | | (7.46%) | |
| Hepatic Steatosis | 1/46 | 1 | 1/65 | 1 | 3/67 | 3 |
| | (2.17%) | | (1.54%) | | (4.48%) | |

Infections and infestations

| | | | | | | |
|---|---|---|---|---|---|---|
| Genital Herpes Simplex | 0/46 | 0 | 1/65 | 1 | 0/67 | 0 |
| | (0%) | | (1.54%) | | (0%) | |
| Influenza | 1/46 | 1 | 3/65 | 3 | 2/67 | 2 |
| | (2.17%) | | (4.62%) | | (2.99%) | |
| Nasopharyngitis | 7/46 | 9 | 1/65 | 1 | 8/67 | 10 |
| | (15.22%) | | (1.54%) | | (11.94%) | |
| Tinea Pedis | 0/46 | 0 | 2/65 | 2 | 0/67 | 0 |
| | (0%) | | (3.08%) | | (0%) | |
| Upper Respiratory Tract | 2/46 | 2 | 4/65 | 5 | 6/67 | 7 |
| Infection | (4.35%) | | (6.15%) | | (8.96%) | |

Injury, poisoning and procedural complications

| | | | | | | |
|---|---|---|---|---|---|---|
| Arthropod Bite | 0/46 | 0 | 1/65 | 1 | 1/67 | 1 |
| | (0%) | | (1.54%) | | (1.49%) | |
| Buttock Injury | 0/46 | 0 | 1/65 | 1 | 0/67 | 0 |
| | (0%) | | (1.54%) | | (0%) | |
| Fall | 0/46 | 0 | 2/65 | 2 | 0/67 | 0 |
| | (0%) | | (3.08%) | | (0%) | |

Investigations

| | | | | | | |
|---|---|---|---|---|---|---|
| Alanine | 15/46 | 19 | 1/65 | 3 | 18/67 | 22 |
| Aminotransferase | (32.61%) | | (1.54%) | | (26.87%) | |
| Increased | | | | | | |
| Aspartate | 3/46 | 4 | 1/65 | 1 | 4/67 | 7 |
| Aminotransferase | (6.52%) | | (1.54%) | | (5.97%) | |
| Increased | | | | | | |
| Fibrin D Dimer | 2/46 | 2 | 0/65 | 0 | 5/67 | 5 |
| Increased | (4.35%) | | (0%) | | (7.46%) | |
| Lymphocyte Count | 0/46 | 0 | 0/65 | 0 | 0/67 | 0 |
| Increased | (0%) | | (0%) | | (0%) | |
| Neutrophil Count | 0/46 | 0 | 0/65 | 0 | 0/67 | 0 |
| Decreased | (0%) | | (0%) | | (0%) | |
| White Blood Cell Count | 0/46 | 0 | 0/65 | 0 | 0/67 | 0 |
| Decreased | (0%) | | (0%) | | (0%) | |

Metabolism and nutrition disorders

| | | | | | | |
|---|---|---|---|---|---|---|
| Iron Deficiency | 0/46 | 0 | 1/65 | 1 | 0/67 | 0 |
| | (0%) | | (1.54%) | | (0%) | |

Musculoskeletal and connective tissue disorders

| | | | | | | |
|---|---|---|---|---|---|---|
| Arthralgia | 4/46 | 8 | 4/65 | 5 | 5/67 | 9 |
| | (8.7%) | | (6.15%) | | (7.46%) | |
| Joint Swelling | 1/46 | 1 | 1/65 | 1 | 1/67 | 1 |
| | (2.17%) | | (1.54%) | | (1.49%) | |
| Synovitis | 0/46 | 0 | 1/65 | 1 | 1/67 | 1 |
| | (0%) | | (1.54%) | | (1.49%) | |

Nervous system disorders

| | | | | | | |
|---|---|---|---|---|---|---|
| Headache | 0/46 | 0 | 3/65 | 3 | 1/67 | 2 |
| | (0%) | | (4.62%) | | (1.49%) | |

Respiratory, thoracic and mediastinal disorders

| | | | | | | |
|---|---|---|---|---|---|---|
| Cough | 2/46 | 2 | 0/65 | 0 | 4/67 | 5 |
| | (4.35%) | | (0%) | | (5.97%) | |

Skin and subcutaneous tissue disorders

| | | | | | | |
|---|---|---|---|---|---|---|
| Dermatitis Allergic | 0/46 | 0 | 1/65 | 1 | 0/67 | 0 |
| | (0%) | | (1.54%) | | (0%) | |
| Pruritus | 0/46 | 0 | 1/66 | 1 | 0/67 | 0 |
| | (0%) | | (1.54%) | | (0%) | |

TABLE 21
Other Adverse Events in Patients Treated with Fitusiran 50 mg Q2M

| | Cohort A: SAS 2 - | | Cohort A: SAS 2 - | |
|---|---|---|---|---|
| | BPA Prophylaxis | | Fitusiran 50 mg Q2M | |
| | Affected/At | | Affected/At | |
| | Risk (%) | # Events | Risk (%) | # Events |
| Total | 1/2 (50%) | | 2/2 (100%) | |

Blood and lymphatic system disorders

| | | | | |
|---|---|---|---|---|
| Anaemia | 0/2 (0%) | 0 | 0/2 (0%) | 0 |

Gastrointestinal disorders

| | | | | |
|---|---|---|---|---|
| Abdominal Pain | 0/2 (0%) | 0 | 0/2 (0%) | 0 |
| Dental Caries | 0/2 (0%) | 0 | 0/2 (0%) | 0 |
| Eosinophilic Oesophagitis | 0/2 (0%) | 0 | 0/2 (0%) | 0 |
| Gastrointestinal Motility Disorder | 0/2 (0%) | 0 | 0/2 (0%) | 0 |
| Haemorrhoids | 1/2 (50%) | 1 | 0/2 (0%) | 0 |
| Nausea | 0/2 (0%) | 0 | 0/2 (0%) | 0 |
| Teething | 0/2 (0%) | 0 | 0/2 (0%) | 0 |
| Toothache | 0/2 (0%) | 0 | 2/2 (100%) | 2 |
| Vomiting | 0/2 (0%) | 0 | 0/2 (0%) | 0 |

General disorders

| | | | | |
|---|---|---|---|---|
| Injection Site Erythema | 0/2 (0%) | 0 | 0/2 (0%) | 0 |
| Injection Site Pain | 0/2 (0%) | 0 | 0/2 (0%) | 0 |

Hepatobiliary disorders

| | | | | |
|---|---|---|---|---|
| Cholecystitis | 0/2 (0%) | 0 | 0/2 (0%) | 0 |
| Cholelithiasis | 0/2 (0%) | 0 | 0/2 (0%) | 0 |
| Hepatic Steatosis | 0/2 (0%) | 0 | 0/2 (0%) | 0 |

Infections and infestations

| | | | | |
|---|---|---|---|---|
| Genital Herpes Simplex | 0/2 (0%) | 0 | 0/2 (0%) | 0 |
| Influenza | 0/2 (0%) | 0 | 0/2 (0%) | 0 |
| Nasopharyngitis | 0/2 (0%) | 0 | 0/2 (0%) | 0 |
| Tinea Pedis | 0/2 (0%) | 0 | 0/2 (0%) | 0 |
| Upper Respiratory Tract Infection | 0/2 (0%) | 0 | 0/2 (0%) | 0 |

Injury, poisoning and procedural complications

| | | | | |
|---|---|---|---|---|
| Arthropod Bite | 0/2 (0%) | 0 | 0/2 (0%) | 0 |
| Buttock Injury | 0/2 (0%) | 0 | 0/2 (0%) | 0 |
| Fall | 0/2 (0%) | 0 | 0/2 (0%) | 0 |

Investigations

| | | | | |
|---|---|---|---|---|
| Alanine Aminotransferase Increased | 0/2 (0%) | 0 | 0/2 (0%) | 0 |
| Aspartate Aminotransferase Increased | 0/2 (0%) | 0 | 0/2 (0%) | 0 |
| Fibrin D Dimer Increased | 0/2 (0%) | 0 | 1/2 (50%) | 1 |
| Lymphocyte Count Increased | 1/2 (50%) | 1 | 0/2 (0%) | 0 |
| Neutrophil Count Decreased | 1/2 (50%) | 1 | 0/2 (0%) | 0 |
| White Blood Cell Count Decreased | 1/2 (50%) | 1 | 0/2 (0%) | 0 |

Metabolism and nutrition disorders

| | | | | |
|---|---|---|---|---|
| Iron Deficiency | 0/2 (0%) | 0 | 0/2 (0%) | 0 |

Musculoskeletal and connective tissue disorders

| | | | | |
|---|---|---|---|---|
| Arthralgia | 0/2 (0%) | 0 | 0/2 (0%) | 0 |
| Joint Swelling | 0/2 (0%) | 0 | 0/2 (0%) | 0 |
| Synovitis | 0/2 (0%) | 0 | 0/2 (0%) | 0 |

Nervous system disorders

| | | | | |
|---|---|---|---|---|
| Headache | 0/2 (0%) | 0 | 0/2 (09%) | 0 |

Respiratory, thoracic and mediastinal disorders

| | | | | |
|---|---|---|---|---|
| Cough | 0/2 (0%) | 0 | 0/2 (0%) | 0 |

Skin and subcutaneous tissue disorders

| | | | | |
|---|---|---|---|---|
| Dermatitis Allergic | 0/2 (0%) | 0 | 0/2 (0%) | 0 |
| Pruritus | 0/2 (0%) | 0 | 0/2 (0%) | 0 |

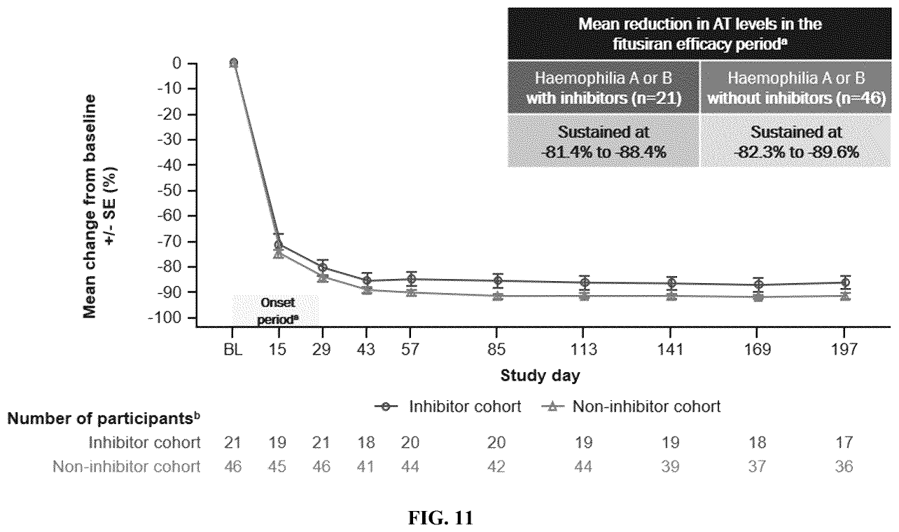

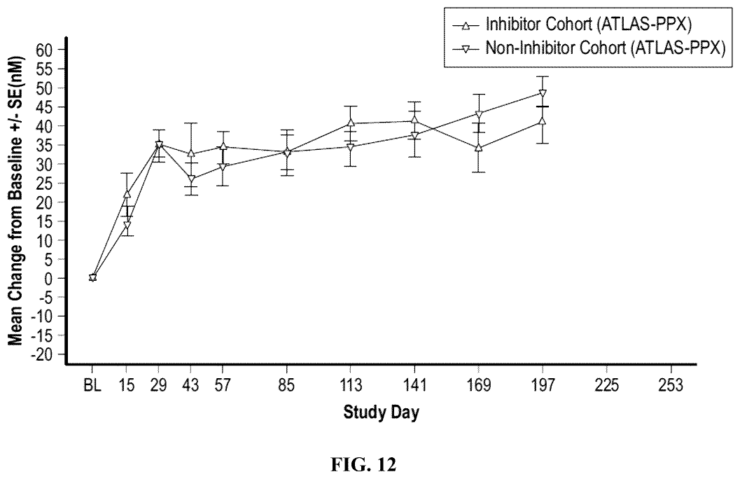

Antithrombin Levels: Reduced AT levels were observed in patients during the fitusiran efficacy period (FIG. 11). In patients with inhibitors, there was a mean reduction in AT levels from baseline by 81.4% at day 29. In patients without inhibitors, there was a mean reduction in AT levels from baseline by 82.3% at day 29. AT levels remained reduced during the entire study period. In patients with inhibitors, there was a mean increase in peak thrombin generation (TG) from baseline of 35.0 nM on day 29. In patients without inhibitors, there was a mean increase in peak TG from baseline of 34.9 nM at day 29. TG remained elevated during the entire study period in all three trials (FIG. 12).

Adolescent Subgroup Analysis

19 adolescents (12-17 years) were enrolled. The baseline characteristics of the adolescents enrolled in the trial are shown in Table 22.

TABLE 22
Characteristics of the Adolescent Patients

| | Inhibitor | | Non-inhibitor | |
|---|---|---|---|---|
| Characteristic | (n = 7) | | (n = 12) | |
| Age, years, mean (SD) | 14.0 | (2.0) | 14.7 | (1.2) |
| Weight, kg, mean (SD) | 68.9 | (18.7) | 67.2 | (11.4) |
| Hemophilia type, n (%) | | | | |
| Hemophilia A | 4 | (57.1) | 8 | (66.7) |
| Hemophilia B | 3 | (42.9) | 4 | (33.3) |
| Bleeding episodes in the last six | 4.0 | (2.0; 6.0) | 2.0¹ | (1.5; 3.5) |
| months prior to screening, median | | | | |
| (IQR) | | | | |
| Target joint at baseline, median | 0.0 | (0.0; 1.0) | 0.0 | (0.0, 0.0) |
| (IQR) | | | | |
| Haemo-QOL total transformed | 53.4 | (8.0) | 30.1 | (20.8) |
| score at baseline², mean (SD) | | | | |

¹Number of bleeding events in the 12 months prior to screening was recorded for non-inhibitor participants.
²Inhibitor n = 6, non-inhibitor n = 11.

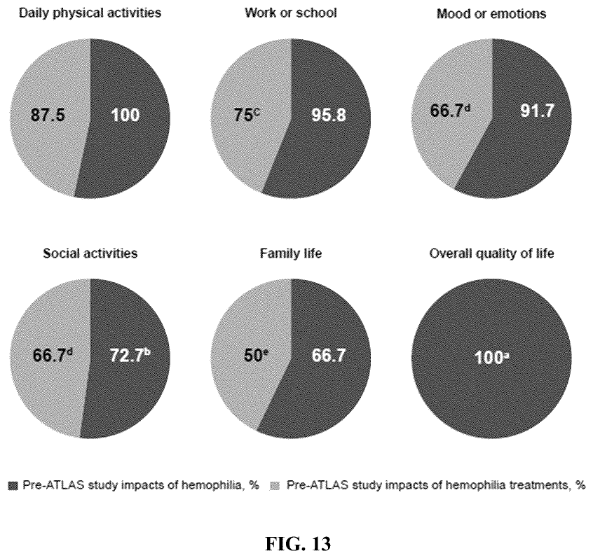

Median ABR was 0.00 in the fitusiran arm compared to 2.2 in the CFC/BPA arm. The median observed ABR, AjBR, and AsBR for adolescent patients enrolled in the trial are shown in FIG. 13.

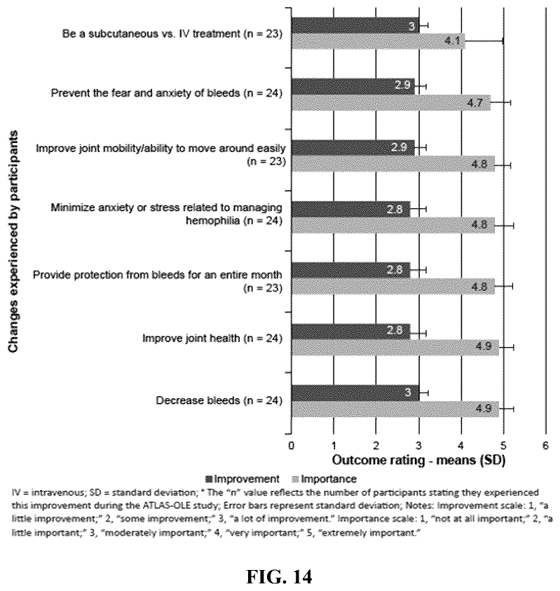

Fitusiran prophylaxis improved HRQoL with a mean change in transformed total score from baseline to month 9 of −4.3 in the CFC/BPA prophylaxis period compared to −6.3 over the full trial period as measured using the Haemo-QoL survey (FIG. 14). The safety profile of fitusiran in adolescents was consistent with that observed in adult participants.

In adolescents with hemophilia A or B, with or without inhibitors, fitusiran prophylaxis demonstrated an improvement in bleeding phenotype. Decreases in Haemo-QoL scores support broad improvements in HRQoL with fitusiran prophylaxis compared with CFC and BPA treatments. The benefit/risk assessment was favorable.

### CONCLUSIONS

Fitusiran 80 mg QM prophylaxis achieved a highly significant 61.1% (95% CI, 32.5% to 77.6%) ABR reduction during the efficacy period in participants with hemophilia A or B with or without inhibitors who switched from their prior prophylaxis with factor or BPA (P<0.001). The observed median (IQR) ABRs were lower during the fitusiran efficacy period than during the factor/BPA prophylaxis period (0.00 [0.00; 2.25] and 4.35 [2.17; 10.87]).

63.1% of participants experienced zero treated bleeds during the fitusiran efficacy period compared to 16.9% during the factor or BPA prophylaxis period. These findings were supported by substantially lower rates of other bleeding-related end points (events of spontaneous bleeding, joint bleeding) during fitusiran prophylaxis compared to factor or BPA prophylaxis. These results demonstrate fitusiran 80 mg monthly prophylaxis provided a significant level of protection against bleeding in participants with hemophilia A or B with or without inhibitors. Fitusiran prophylaxis significantly improved health-related quality of life as measured by Haem-A-QOL total score. Nominal results suggest an improvement in the physical health domain subdomain. Reported AEs were generally consistent with the previously identified risks of fitusiran. The safety and efficacy of a revised dose and regimen are currently being evaluated in ongoing clinical studies.

In conclusion, once-monthly fitusiran prophylaxis significantly reduced bleeding versus factor/BPA prophylaxis with a median ABR of zero in patients with haemophilia A or B with and without inhibitors, resulting in a meaningful improvement in HRQoL. Reported AEs were generally consistent with the previously identified risks of fitusiran. Thus, these results support that fitusiran may provide a monthly, subcutaneous, prophylactic therapeutic option for patients with hemophilia A or B with or without inhibitors previously receiving factor concentrate or BPA prophylaxis. In addition, fitusiran prophylaxis significantly reduced bleeding rates compared to prior factor/BPA prophylaxis, with zero bleed rates of >60%, indicating that hemostatic levels of efficacy were achieved with fitusiran prophylaxis. Fewer bleeds resulted in lower factor/BPA consumption with fitusiran prophylaxis compared to factor/BPA prophylaxis, with fewer injections and lower doses of factor/BPA required to treat each bleed. The subcutaneous, relatively infrequent administration route of fitusiran prophylaxis could reduce the overall treatment and disease burden, improve QoL, and increase adherence for patients with hemophilia A or B, with or without inhibitors, compared to factor/BPA prophylaxis.

### Example 3: Qualitative Semi-Structured Interviews of Participants of ATLAS-OLE Trial

In addition to the clinical trial described in Examples 1 and 2 (ATLAS-PPX), fitusiran has been evaluated in two additional Phase 3 trials: ATLAS-INH (a study of patients with inhibitors) and ATLAS-A/B (a study of patients without inhibitors). After completing any of these three studies, patients were eligible to participate in an open-label extension (OLE) study (ATLAS-OLE).

This Example relates to semi-structured interview of patients located in the U.S. and India enrolled in the OLE study. The objective was to better understand patients' and caregivers' experiences with hemophilia A or B (with or without inhibitors) and its treatments, including fitusiran, as well as their perceptions of—and satisfaction with—fitusiran during the OLE trial.

The interviewees included patients 18 years of age. In the case of patients that were cognitively unable to participate in the interview, or patients between 12 and 18 years of age, caregivers were interviewed. All patients were interviewed at least one month after receiving their second fitusiran dose in the OLE trial.

The one-hour semi-structured interviews were conducted over the telephone. The audio was recorded and transcribed. Each interview began with targeted open-ended questions to participants pertaining to their experiences with hemophilia and its treatment prior entering the ATLAS program and their perceptions of the impact of hemophilia and its treatment on their daily lives, as well as treatment expectations. The interview discussion was then focused on the participants' experiences during the OLE study, including any changes and benefits that participants noticed. A five-point rating scale was used to rate different attributes of a hypothetical hemophilia treatment and a three-point rating scale was used to rate the degree of improvement noted in these aspects, if any, during the OLE trial. Statistical analysis of the qualitative interview data was performed using anonymized interview transcripts and ATLAS.ti 9 software. Descriptive demographic and clinical information were summarized for the overall sample, and are shown in Table 23.

TABLE 23
Characteristics of the Participants

| Characteristic | Total (N = 24) |
|---|---|
| Patient, n (%) | 21 (87.5) |
| Caregiver, n (%) | 3 (12.5) |

| Participant age at screening | Mean (SD) | 27.0 | (8.9) |
|---|---|---|---|
| (years)^{a} | Range | 13-49 | |
| Country of residence, n (%) | India | 23 | (95.8) |
| | United States | 1 | (4.2) |
| Hemophilia type, n %) | Hemophilia A | 18 | (75.0) |
| | Hemophilia B | 6 | (25.0) |
| History of inhibitors prior to | No | 14 | (58.3) |
| ATLAS studies, n (%) | Yes | 10 | (41.7) |
| Previous hemophilia | CFC | 20 | (83.3) |
| treatments, n (%)^{b} | aPCC | 8 | (33.3) |

aPCC, activated prothrombin complex concentrate; SD, standard deviation; CFC, clotting factor concentrate.
Note:
Participant age at screening, hemophilia type, and inhibitor status were from Sanofi's clinical study data. Other data reported in this table were provided by the participant during the interview;
^{a}Includes ages of 3 minor patients whose caregiver participated (<18 years of age at time of interview, n = 2; or were cognitively unable to participate, n = 1). One parent (i.e., caregiver) of each patient was interviewed
instead;
^{b}Some participants reported experience with multiple treatments for their hemophilia in the past.

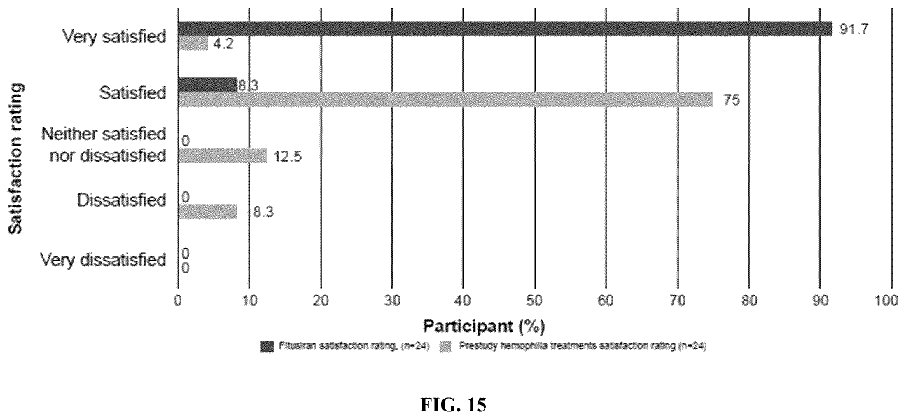

A total of 24 participants were interviewed. The mean (SD) age of participants was 27 (8.9) years. As shown in FIG. 15, prior to enrolment in the fitusiran clinical trials, participants stated their (or their child's) hemophilia-related bleeds (joint and muscle), pain, weakness, inflexible joints, and swelling significantly and negatively impacted their daily lives in a number of areas. Almost all participants (n=21; 87.5%) reported hemophilia treatments impacted their daily physical activities, followed by impacts to work or school.

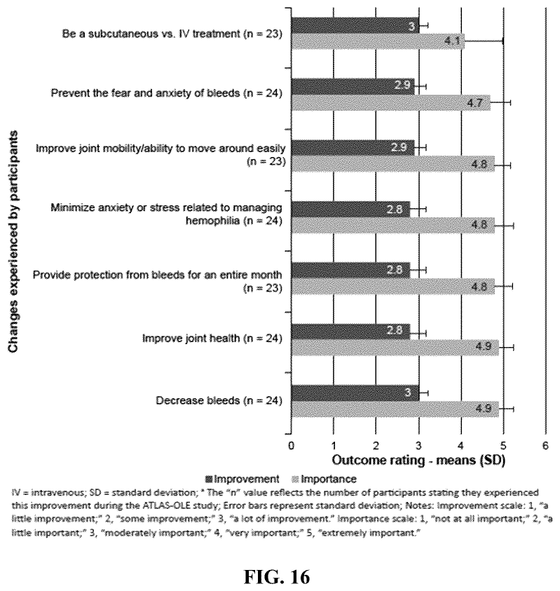

FIG. 16 shows that participants rated “decreased bleeds” as the most important attributes from a hemophilia treatment and as one of the top two items for which they experienced most of improvement during the OLE trial. Other highly important attributes for a hypothetical hemophilia treatment included “improved joint health”, “improved joint mobility/ability to move around easily”, “protection from bleeds for an entire month”, and “minimize anxiety or stress related to managing hemophilia.” All participants reported that improvements they observed during the ATLAS-OLE trial positively impacted their quality of life, and their ability and confidence to participate daily physical activities, enjoy a family life, participate in social activities, and improve their general mood or emotions (Table 24). Most participants (85%) also reported positive impact of improvements at work or school.

TABLE 24
Improvement in Participants' Quality of Life

| Impact of Improvement | Total (N = 24) n (%) | |
|---|---|---|
| Daily physical activities | 24 | (100.0) |
| Family Life | 22 | (100.0)^{a} |
| Social Activities | 20 | (100.0)^{b} |
| Overall Quality of Life | 18 | (100.0)^{c} |
| Feeling Safer | 23 | (100.0)^{d} |
| Work or School | 17 | (85.0)^{b} |
| Mood or Emotions | 15 | (100.0)^{e} |

^{a}Percentage based on the 22 participants asked this question;
^{b}Percentage based on the 20 participants asked this question;
^{c}Percentage based on the 18 participants asked this question;
^{d}Percentage based on the 23 participants asked this question;
^{e}Percentage based on the 15 participants asked this question.

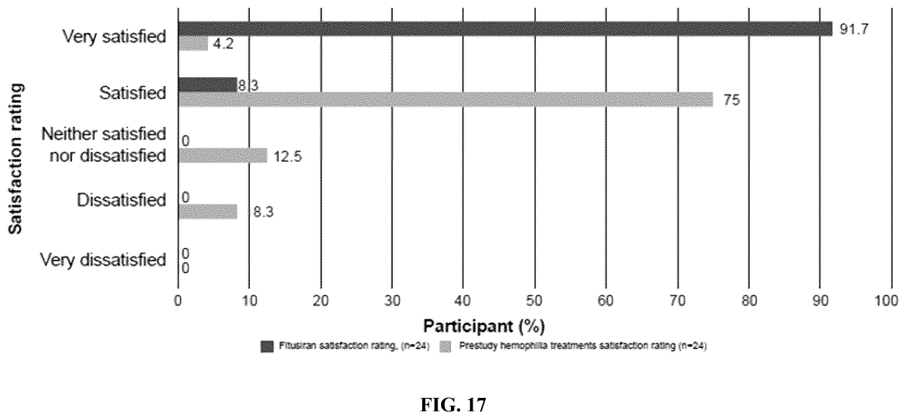

FIG. 17 shows that, while 75% of participants were “satisfied” with their previous treatment prior to recruitment in the ATLAS trial, nearly 92% of them were “very satisfied” with fitusiran, especially with regards to its ability to prevent bleeds, its duration of effects, and its convenience. Additionally, nearly all (23/24) participants preferred fitusiran prophylaxis over their previous hemophilia treatment.

Among the quotes recorded during the interviews, participants reported for example that “After I took fitusiran, I became like a normal person.” “. . . I can walk normal” and that” . . . [I am] “ . . . very confident because if I get the fitusiran I can say no bleed will happen.”

In conclusion, fitusiran reaches the treatment expectations of patients with hemophilia and their caregivers. Nearly all participants were very satisfied with fitusiran therapy and preferred fitusiran over prior therapy. All participants treated with fitusiran reported positive changes on daily physical activities, family life, social activities, feeling safer, and mood. These findings reflect the impact on participants' quality of life that fitusiran prophylaxis provides by improving the bleeding phenotype.

Nearly all participants were very satisfied with fitusiran therapy and preferred fitusiran over prior therapy. This qualitative study provides valuable and positive insights about patients' and caregivers' experiences with hemophilia and its treatment.

1. A method of reducing the annual bleeding rate (ABR) in a human subject having hemophilia A or B with or without inhibitors who has been on prophylactic treatment with a replacement factor or a bypassing agent (BPA), comprising
subcutaneously administering to the human subject in need thereof a therapeutically effective amount of fitusiran, and
terminating the prophylactic replacement factor or BPA treatment in the subject within about two months, about one month, or optionally within about 28 or about seven days, of the first dose of fitusiran.

2. (canceled)

3. (canceled)

4. The method of claim 1, wherein the administration reduces the median ABR of the subject to two or less, one or less, or zero, further optionally wherein the historical ABR of the subject is greater than 4.

5. A method of reducing the annual spontaneous bleeding rate (AsBR) in a human subject having hemophilia A or B with or without inhibitors who has been on prophylactic treatment with a replacement factor or a bypassing agent (BPA), comprising
subcutaneously administering to the human subject in need thereof a therapeutically effective amount of fitusiran, and
terminating the prophylactic replacement factor or BPA treatment in the subject within one month, optionally within about 28 or about seven days, of the first dose of fitusiran.

6. (canceled)

7. (canceled)

8. The method of claim 5, wherein the administration reduces AsBR of the subject to one or less, or zero, optionally wherein the historical AsBR of the subject is greater than 2.

9. A method of reducing the annualized joint bleeding rate (AjBR) in a human subject having hemophilia A or B with or without inhibitors who has been on prophylactic treatment with a replacement factor or a bypassing agent (BPA), comprising
subcutaneously administering to the human subject in need thereof a therapeutically effective amount of fitusiran, and
terminating the prophylactic replacement factor or BPA treatment in the subject within about one month, optionally within about 28 or about seven days, of the first dose of fitusiran.

10. (canceled)

11. (canceled)

12. The method of claim 9, wherein the administration reduces the AjBR of the subject to one or less, or zero, optionally wherein the historical AjBR of the subject is greater than 2.

13. A method of improving patient-reported outcome (PRO) in a human subject having hemophilia A or B with or without inhibitors who has been on prophylactic treatment with a replacement factor or a bypassing agent (BPA), comprising
subcutaneously administering to the human subject in need thereof a therapeutically effective amount of fitusiran, and
terminating the prophylactic replacement factor or BPA treatment in the subject within about one month, optionally within about 28 or about seven days, of the first dose of fitusiran, optionally wherein the PRO is improved in one or more quality of life (QoL) domains.

14. (canceled)

15. A method of improving quality of life (QoL) in a human subject having hemophilia A or with or without inhibitors who has been on prophylactic treatment with a replacement factor or a bypassing agent (BPA), comprising
subcutaneously administering to the human subject in need thereof a therapeutically effective amount of fitusiran, and
terminating the prophylactic replacement factor or BPA treatment in the subject within about one month, optionally within about 28 or about seven days, of the first dose of fitusiran,
optionally wherein the QoL is improved in one or more QoL domains.

16. (canceled)

17. The method of claim 13, wherein the one or more QoL domains are domains in a QoL questionnaire, optionally wherein the QoL questionnaire is Haemophilia Quality of Life Questionnaire for Adults (Haem-A-QoL).

18. (canceled)

19. The method of claim 13, wherein the one or more QoL domains are domains in a QoL questionnaire, optionally wherein the QoL questionnaire is Hemophilia Activities List (HAL).

20. (canceled)

21. The method of claim 13, wherein the one or more QoL domains are domains in a QoL questionnaire, optionally wherein the QoL questionnaire is Treatment Satisfaction Questionnaire for Medication Version 9 (TSQM-9).

22. (canceled)

23. The method of claim 13, wherein the one or more QoL domains are domains in a QoL questionnaire, optionally wherein the QoL questionnaire is EuroQol 5-Dimensions (EQ-5D-5L).

24. (canceled)

25. The method of claim 13, wherein the one or more QoL domains are domains in a QoL questionnaire, optionally wherein the QoL questionnaire is Haemophilia Quality of Life Questionnaire for children and adolescents (Haemo-QoL).

26. (canceled)

27. The method of claim 1, wherein the dose of fitusiran is administered to the subject about once a month or about every four weeks or about once every other month or about once every eight weeks.

28. The method of claim 1, wherein the therapeutically effective amount of fitusiran administered to the subject is about 10 to about 100 mg, optionally wherein the therapeutically effective amount is about 80 mg, about 50 mg, about 20 mg, or about 10 mg.

29. The method of claim 1, wherein the subject is a hemophilia A patient with inhibitors or a hemophilia B patient with inhibitors.

30-32. (canceled)

33. The method of claim 1, wherein the subject is a hemophilia A patient without inhibitors or a hemophilia B patient without inhibitors.

34-36. (canceled)

37. The method of claim 1, wherein the subcutaneously administering step comprises administering fitusiran at about 50 mg about every two months or about every eight weeks.

38. The method of claim 37, further comprising:
obtaining a measurement of an antithrombin (AT) level at a steady state in the patient; and
performing one of the following steps:
(i) if the AT level is 15-35%, repeating administration of fitusiran at about 50 mg;
(ii) if the AT level is >35%, subcutaneously administering to the patient fitusiran at about 80 mg about every two months or about every eight weeks, or at about 50 mg about every month or about every four weeks, or
(iii) if the AT level is <15%, discontinuing or pausing fitusiran treatment.

39. The method of claim 1, wherein the subcutaneously administering step comprises administering fitusiran at about 80 mg about every two months or about every eight weeks.

40. The method of claim 1, wherein the subcutaneously administering step comprises administering fitusiran at about 50 mg about every month or about every four weeks.

41. The method of claim 1, wherein the subcutaneously administering step comprises administering fitusiran at about 80 mg about every month or about every four weeks.

42. The method of claim 1, wherein the patient does not have
(i) clinically significant liver disease,
(ii) ALT>1.5×upper limit of normal reference range (ULN),
(iii) AST>1.5×upper limit of normal reference range (ULN),
(iv) hepatitis C,
(v) hepatitis A,
(vi) hepatitis E, and/or
(vii) hepatitis B.

43. The method of claim 1, wherein the patient is an adult or adolescent patient twelve years or older with hemophilia A or B with or without inhibitors.

44. The method of claim 1, wherein the method reduces the annualized weight-adjusted consumption of replacement factor or BPA in the patient.

45. The method of claim 1, wherein the method reduces
(i) the number of breakthrough bleeds requiring treatment in the patient over a given period of time;
(ii) the total weight-adjusted dose of replacement factor/BPA in the patient over a given period of time,
(iii) the mean consumption of replacement factor/BPA in the patient over a given period of time, or
(iv) the number of injections of replacement factor/BPA required to treat a breakthrough bleed in the patient.

46-48. (canceled)

49. A method of:
reducing the annual bleeding rate (ABR),
reducing the annual spontaneous bleeding rate (AsBR),
reducing the annualized joint bleeding rate (AjBR),
improving patient-reported outcome (PRO), or
improving quality of life (QoL)
in a human subject having hemophilia A or B with or without inhibitors who has been on prophylactic treatment with a replacement factor or a bypassing agent (BPA), comprising subcutaneously administering to the human subject in need thereof a therapeutically effective amount of fitusiran.
